(19) United States
(12) Patent Application Publication
(10) Pub. No.: US 2016/0184274 A1
(43) Pub. Date: Jun. 30, 2016

(54) Methods for inhibiting mesenchymal phenotype after epithelial-to-mesenchymal transition

(71) Applicants: Fibrostatin, S.L., Valencia (ES); Universitat de Valencia, Valencia (ES)

(72) Inventors: Juan Saus, Valencia (ES); Fernando Revert, Valencia (ES); Aida Artigot, Valencia (ES); Francisco Revert-Ros, Valencia (ES); Ernesto Lopez-Pascual, Valencia (ES); Raul Blasco, Valencia (ES); Nuria Roda, Valencia (ES); Juan F. Sanz-Cervera, Valencia (ES); Roberto Gozalbo-Rovira, Valencia (ES)

(21) Appl. No.: 14/983,679

(22) Filed: Dec. 30, 2015

Related U.S. Application Data
(60) Provisional application No. 62/098,770, filed on Dec. 31, 2014.
(60) Provisional application No. 62/142,841, filed on Apr. 3, 2015.

(51) Int. Cl. A61K 31/4188; A61K 31/192; G01N 33/574; C07K 16/18

(52) U.S. Cl. CPC A61K 31/4188; C07K 16/18; A61K 31/192; G01N 33/57496; G01N 2333/912; G01N 2458/00

(57) ABSTRACT

Methods of using inhibitors of Goodpasture Antigen Binding Protein for inhibiting mesenchymal phenotype after epithelial-to-mesenchymal transition (EMT), treating an invasive tumor, and detecting EMT in a tissue are described.

### CROSS REFERENCE

This application claims priority to U.S. Provisional Patent Application Ser. Nos. 62/098,770 filed Dec. 31, 2014 and 62/142,841 filed Apr. 3, 2015, incorporated by reference herein in their entirety.

### BACKGROUND

The conformation of the non-collagenous (NC1) domain of the α3 chain of the basement membrane collagen IV (α3NC1) depends in part on phosphorylation. Goodpasture antigen-binding protein (GPBP) (WO 00/50607; WO 02/061430) is a novel non-conventional protein kinase that catalyzes the conformational isomerization of the α3NC1 domain during its supramolecular assembly, resulting in the production and stabilization of multiple α3NC1 conformers in basement membranes. Elevated levels of GPBP have been associated with the production of non-tolerized α3NC1 conformers, which conduct the autoimmune response mediating Goodpasture (GP) disease. In GP patients, autoantibodies against the α3NC1 (also known as GP antigen) cause a rapidly progressive glomerulonephritis and often lung hemorrhage, the two cardinal clinical manifestations of the GP disease.

GPBP (also known as GPBP-1 or 77 kD GPBP) is the primary product of COL4A3BP which undergoes secretion and can be found circulating or associated with collagen IV. The gene also expresses two alternative isoforms, GPBP-2 (also known as GPBPΔ26 or CERT) which remains cytosolic and GPBP-3 (also known as 91 kD GPBP) which associated with cellular membranes and promotes GPBP secretion (WO 00/50607; WO 2010/009856; Revert-Ros et al., 2011, J Biol. Chem 286, 35030-35043). Elevated GPBP expression and secretion have been also associated with collagen IV expansion in immune complex-mediated glomerulonephritis (Revert et al. 2007, Am J Path. 171, 1419-30.).

GPBP yields trimeric and multimeric aggregates, the latter displaying increased specific activity (WO 00/50607). An isolated peptide (Q2) encompassing a five-residue motif which is critical for GPBP multimer stabilization inhibited GPBP kinase activity and abated collagen accumulation in mouse models of immune complex-mediated glomerulonephritis (WO 2004/070025).

Differentiated epithelial cells have the potential to acquire a mesenchymal phenotype through complex biological processes known as epithelial-mesenchymal transition (EMT). Throughout EMT epithelial cells undergo trans-differentiation towards a phenotype with an enhanced migratory capacity and invasiveness, high resistance to apoptosis and an outstanding capacity to synthesize extracellular matrix (see for review Kalluri et al., 2009, J. Clin. Invest. 119:1420-8). Whereas different EMTs have been recognized in embryo implantation and development (type 1); tissue repair and organ fibrosis (type 2); or cancer malignancy and metastasis formation (type 3), the general consensus is that common molecular mechanism must exist among them. Accordingly, E-cadherin expression supports cell-cell attachment in epithelial phenotype and vimentin expression renders cells prone to cell-cell detachment and migration in mesenchymal phenotype. Collagen IV is a primary component of the extracellular matrix that interacts with cancer stem cells (CSCs) forming a protective shield against conventional anti-tumor therapies (Ye J et al., 2014, Tumour Biol. 35, 3945-51; Su C et al., 2007, Cancer Invest. 2, 542-9).

### SUMMARY OF THE INVENTION

In one aspect, the invention provides methods for inhibiting mesenchymal phenotype after epithelial-to-mesenchymal transition (EMT), or methods for treating an invasive tumor comprising administering to a subject in need thereof an amount effective to inhibit mesenchymal phenotype after EMT, or an amount effective to treat an invasive tumor, of an antibody selective for GPBP, or of a compound of formula:

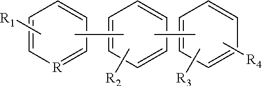

or a pharmaceutically acceptable salt thereof, wherein:
R is selected from N and CR₅;
   R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, or (heteroaryl)C₁-C₆ alkyl;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, —O(CH₂)₁₋₅—C(O)(C₁-C₆ alkoxy), (aryl)C₁-C₆ alkyl, or (heteroaryl)C₁-C₆ alkyl.

In various embodiments, the methods for inhibiting mesenchymal phenotype after EMT may comprise treating a subject with chronic kidney disease, immune-complex mediated glomerulonephritis, organ fibrosis, pulmonary fibrosis, rheumatoid arthritis, or an invasive tumor.

In one embodiment of the treatment methods, the subject has an altered expression of cell markers in a relevant tissue sample compared to a control tissue sample, wherein the altered expression is indicative of an epithelial-to-mesenchymal phenotype transition. In various embodiments, the cell markers include but are not limited to one or more of vimentin, E-cadherin, collagens I and IV, matrix metalloproteinase 9 (MMP-9), chemokine (C—C motif) ligand 2 (CCL2) also referred to as monocyte chemotactic protein 1 (MCP-1), α5 (IV) chain, (α5 (IV))₃ protomer, and Goodpasture antigen binding protein (GPBP). In another embodiment, the subject has an increase in vimentin expression and a decrease in E-cadherin expression in a relevant tissue sample compared to an epithelial cell control.

In one embodiment of the treatment methods of the invention, the subject has an increased expression of α5(IV) chain, and/or (α5 (IV))₃ protomer in a relevant tissue sample compared to a control tissue sample, wherein the increase expression is indicative of an epithelial-to-mesenchymal phenotype transition and/or an invasive tumor phenotype. In a further embodiment, the subject also has an increased expression of (α1)₂α2 (IV) protomer and/or an increased expression α1,α2 (IV) chains in a relevant tissue sample compared to a control tissue sample, wherein the increase expression is indicative of an epithelial-to-mesenchymal phenotype transition and/or an invasive tumor phenotype.

In embodiments of the methods for treating an invasive tumor, the invasive tumor is an invasive carcinoma, including but not limited to invasive breast tumors and invasive lung tumors. In a further embodiment, treating the invasive tumor reduces tumor metastases in the subject.

In a further aspect, the invention provides methods for detecting EMT in a tissue, comprising

(a) contacting a tissue in a subject with an amount effective to label the tissue of a detectably labeled compound of formula:

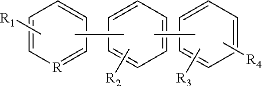

or a pharmaceutically acceptable salt thereof, wherein:
R is selected from N and CR₅;
   R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, or (heteroaryl)C₁-C₆ alkyl;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, —O(CH₂)₁₋₅—C(O)(C₁-C₆ alkoxy), (aryl)C₁-C₆ alkyl, or (heteroaryl)C₁-C₆ alkyl;
   for a time and under conditions suitable to promote binding of the detectably labeled compound to the tissue; and
   (b) detecting the detectably labeled compound bound to the tissue, thereby detecting EMT in the tissue,

In various embodiments, the tissue may be selected from the group consisting of a tumor, a joint, and tissue from any organ. In one embodiment, the tissue is a kidney, and detecting EMT in the kidney indicates that the subject has chronic kidney disease or immune-complex mediated glomerulonephritis. In another embodiment, the tissue is tissue from any organ, and detecting EMT indicates that the subject has organ fibrosis. In a further embodiment, the tissue is a lung, and detecting EMT in the lung indicates that the subject has pulmonary fibrosis. In another embodiment, the tissue is a joint, and wherein detecting EMT indicates that the subject has rheumatoid arthritis. In one embodiment, the tissue is a tumor, and wherein detecting EMT indicates that the subject has an invasive tumor. For example, the tumor may be an invasive carcinoma, including but not limited to invasive breast tumors and invasive lung tumors.

### BRIEF DESCRIPTION OF THE FIGURES

FIG. 1. Extracellular GPBP is mainly multimeric while intracellular GPBP is predominantly trimeric. FLAG-tagged GPBP was expressed in Sf9 insect cells and purified with anti-FLAG® affinity resin from culture medium (extracellular) and from cell lysates (intracellular). The purified proteins were analyzed by gel filtration chromatography with a SUPERDEX® 200 column (GE Healthcare). Gel filtration chromatograms are shown. Peaks corresponding to multimeric and trimeric material are denoted.

FIG. 2. T12 specifically inhibits multimeric extracellular GPBP. Multimeric extracellular and trimeric intracellular GPBP were purified by affinity chromatography and subsequent gel filtration chromatography, and used for in vitro phosphorylation assays in absence (−) or presence (+) of T12 (50 μM). Reactions were subjected to SDS-PAGE, Western blot onto PVDF membrane and autoradiography, and further protein detected with anti-FLAG primary antibodies. Autoradiography and anti-FLAG® bands were quantified with WCIF IMAGEJ® software, and the normalized kinase activity estimated. Shown are normalized kinase activities. Data were analyzed with Student's t-test using GRAPHPAD® Prism software. As indicated, Mean differences were statistically significant for kinase assays using multimeric extracellular GPBP. ns, non-significant. Samples were analyzed in triplicate and the data represents the Mean (±SEM) of three independent assays.

FIG. 3. Mesenchymal cancer cells secrete more GPBP than epithelial cancer cells and are more sensitive to T12. A, A427 (mesenchymal) and A549 (epithelial) cell cultures were lysed and equal amounts of lysates were analyzed by Western blot with antibodies for E-cadherin (E-Cad), vimentin (Vim) or GAPDH for control loading purposes. B, media from the indicated cultures were immunoprecipitated with agarose beads-conjugated anti-GPBP N26 antibodies and immunoprecipitates were analyzed by Western blot with anti-GPBP N27 antibodies (WO 2010/009856). C, mRNA from A427 cells and A549 spheroids cultured in ultralow binding plates for 2 days was analyzed by reverse transcriptase (RT) coupled to a quantitative polymerase chain reaction (qPCR). Shown are the relative quantities (RQ) of the indicated mRNAs of A427 cells using levels from A549 cells as reference. A427 cells did not express significant amounts of α3(IV), α4(IV) and α6(IV) mRNAs. D, T12 IC50 for the indicated cultures was estimated using ALAMARBLUE® and indicated in the Table. Mesenchymal A427 cells show more sensibility to T12 than epithelial A549 cells.

FIG. 4. T12 inhibits GPBP-induced phenotype transition in A549 cultures. A549 cells expressing either GPBP-EYFP fusion protein or EYFP were cultured during 24 h in the presence (+) or absence (−) of T12 (10 μM.) Then cells were lysed and similar amounts of lysates were analyzed by Western blot with antibodies against the indicated proteins. Loading equivalence was confirmed by tubulin expression analysis in each of the individual experiments (not shown).

FIG. 5. GPBP and collagen IV are upregulated in EMT. A549 spheroids were grown and subjected to EMT induction with TGF-β and TNF-α (Mesenchymal) or left unstimulated (Epithelial) during 4 days, and then fixed and analyzed by immunofluorescence confocal microscopy (lower images) for detection of the α1 and α2 chains of collagen IV with anti-α1 α2(IV) antibodies (COL4, white). Nuclei were visualized with DAPI (grey). Additionally, corresponding phase contrast images of spheroids in culture plates were acquired with an inverted microscope (upper images). In the middle, A549 spheroids were stimulated with TNF-α and TGF-β (TT) for EMT induction or left unstimulated (−) during 24 h. Lysates were prepared and GPBP and E-cadherin expression analyzed by Western blot with specific antibodies. GAPDH was analyzed as loading control. The reduction of E-Cadherin levels is indicative of EMT.

FIG. 6. GPBP and collagen IV stabilize A549 mesenchymal spheroids. A549 cells were transfected with siRNAs targeting the indicated mRNAs or with a negative control siRNA (siCONT) and cultured for 24 h. Cells were subjected to spheroid formation during 2 days, transferred to ultra-low binding plates and cultured unstimulated (Epithelial) or stimulated with TNF-α and TGF-β (Mesenchymal). Cell death over time was assessed by measuring LDH activity in spheroids culture media. Reductions in mRNA levels in spheroids were confirmed at the end of the assays by RT-qPCR (not shown).

FIG. 7. T12 disrupts collagen IV network and reduces A549 mesenchymal spheroids viability. A549 spheroids were subjected to EMT induction with TGF-β and TNF-α during 4 days, and treated with T12 (10 μM) or maintained untreated (Cont) for the last 2 days. In A, spheroids were analyzed by immunofluorescence confocal microscopy with anti-α1α2 (IV) antibodies (COL4, white) and DAPI to visualize the cell nuclei (grey). In B, the culture media of the spheroids in A were used for assessing cell death by measuring LDH activity. Data were represented and analyzed with GRAPHPAD® Prism software. Represented are Means (±SEM). Statistically significant differences were found between mesenchymal T12-treated and not treated spheroids, according to Student's t-test.; ns, non-significant; ** P<0.01.

FIG. 8. Doxorubicin-resistant A549 mesenchymal spheroids overexpress GPBP. Doxorubicin-resistant A549 cells (A549DR) and A549 cells were grown in spheroids and EMT was induced with TGF-β and TNF-α during 4 days. Then spheroids were fixed and analyzed by immunofluorescence confocal microscopy with anti-GPBP antibodies (GPBP, mAb e11-2-FITC) and with anti-α1α2(IV) antibodies to visualize the collagen IV network [anti-α1α2(IV)-AF647]. Acquired images were analyzed with WCIF IMAGEJ®software for detection of points of co-localization of GPBP and COL4 shown in the right.

FIG. 9. T12 enhances intracellular accumulation of doxorubicin in A549 mesenchymal spheroids. A549 spheroids were subjected to EMT induction with TGF-β and TNF-α during 4 days, and treated with T12 (10 μM) or maintained untreated (Placebo) for the last 2 days. Three hours before spheroid analysis doxorubicin (1 μM) was added. Spheroids were fixed and analyzed by immunofluorescence confocal microscopy with anti-α1α2(IV) antibodies for collagen IV network visualization (grey). Doxorubicin was detected by its own auto-fluorescence. Nuclei containing doxorubicin (white) were more abundant in spheroids treated with doxorubicin and T12 than in spheroids treated only with doxorubicin. The intensively stained nuclei were smaller and pyknotic revealing to correspond to dead tumor cells.

FIG. 10. T12 targets tumors with mesenchymal phenotype but requires doxorubicin sensitization to show efficacy on tumors with epithelial phenotype. A, graphs show the Mean relative volume (±SEM) over time of A549 tumors in mice that were treated with the indicated compounds. Eight-week old athymic NMRi-Foxn1nu/Foxn1nu male mice were subcutaneously inoculated with 3×10⁶ A549 cells dispersed in culture media and Matrigel (Corning) (1:1). When tumors reached a 200-300 mm³ volume mice were sorted into four different groups that were either left untreated (Control) or treated with doxorubicin (Doxo, 4 mg/kg/week administered intraperitoneally once weekly), with T12 (20 mg/kg/day diluted in drinking water daily), or with both (T12+Doxo). Tumor dimensions were periodically measured with a caliper and tumor volumes were calculated with the formula Volume=(Length×Width²)/2. At the end of the experiment mice were sacrificed, tumors were dissected, RNA extracted and mRNA levels of E-cadherin and vimentin measured by RT-qPCR to confirm mesenchymal or epithelial tumor phenotype (not shown). Relative tumor volumes were calculated using the tumor volumes at the onset of the treatment period for reference purposes. The number of animals was six per group for assays with mesenchymal tumors and ten per group for assays with epithelial tumors. Data were analyzed with Two-way ANOVA and Dunnet's multiple comparison test using GRAPHPAD® software. Asterisks indicate means with statistically significant differences respect to Control values. *P<0.05; **P<0.01; ***P<0.001. B, 10⁴4 T1 mouse breast cancer cells were inoculated into mammary fat pads of 8-week-old female Balb/c mice and either left untreated (Control) or treated with the indicated compound (T12, 12 mg/kg/day administered in the drinking water). Tumor volumes were calculated at different times after cell inoculation and are shown as Mean (±SEM). Where indicated, differences are statistically significant (****P<0.0001, n=10 in both groups) according to Two-Way ANOVA and Bonferroni test. C, Represented are the Mean number (±SEM) of superficial metastasis per lung identified at the end of the assay (day 25). Differences are statistically significant (**P=0.002) according to Kruskal-Wallis test. Control, n=43; T12, n=18.

FIG. 11. T12 targets circulating 4T1 cancer cells. A, Balb/c female mice were inoculated with 4T1 mouse breast cancer cells at mammary fat pads. At day 20 after cell inoculation mice were subjected to PET analysis for metastases detection. White arrows denote metastatic foci at the spinal cord. B, Balb/c female from A were either left untreated (Control), or were treated with T12 in drinking water from day 20. At day 35 blood was seeded in the presence of 6-thioguanine and circulating 4T1 cells selected and further cultured. Shown are phase contrast images of the resulting cultures acquired with an inverted microscope. Circulating 4T1 cells could not be recovered from T12-treated mice. C, circulating 4T1 cells recovered as in B and 4T1 cells were cultured in standard 2D cultures and subjected to RNA extraction, and RNA samples were analyzed by RT-qPCR. Shown are the relative quantities (RQ) of the indicated mRNAs of circulating 4T1 cells using levels from 4T1 cells as reference. D, the cultures in C were treated with the indicated concentrations of T12 for 24 h. Dead cells in culture media were quantified by measuring cell size using an automated cell counter (Moxi Z, Orflo). The number of dead cells in the culture media of the respective untreated cells (−) was used as reference. E, circulating 4T1 cells recovered as in B or 4T1 cells were cultured in ultra-low binding plates and treated with the indicated concentrations of T12 for 9 days, cells were lysed and analyzed by Western blot using antibodies specific for activated caspase 3. The levels of GAPDH were determined for loading control purposes.

FIG. 12. bioT12 binds to GPBP in A549 tumors. Above, cryosections of A549 xenografts grown in immune-deficient mice were stained with biotinylated T12 (bioT12) and with antibodies against GPBP and collagen IV (COL4), followed by suitable fluorophore-conjugated streptavidin and secondary antibodies, and analyzed by confocal microscopy. Below, acquired images were analyzed with WCIF IMAGEJ®software for detection of points of co-localization between the indicated image pairs of the upper panel. Outputs of co-localization analyses show only points of co-localization.

FIG. 13. bioT12 specifically binds tumors. A, cryosections of tumors of Lewis lung carcinoma (LLC) grown in a C57BL/6 mouse and the indicated normal tissues from an 8-week-old C57BL/6 female mouse were stained with bioT12 and with fluorescein-conjugated streptavidin or with antibodies against GPBP (N27-AF546), and analyzed by confocal microscopy. Nuclei were stained with DAPI. B, shown are the intensity of fluorescence Means (±SEM) of GPBP and bioT12 in the images displayed in A expressed as arbitrary units (AU). fFluorescences were normalized with the Mean of the corresponding fluorescence. Parameters were measured using Adobe ADOBE PHOTOSHOP CS® and analyzed with GRAPHPAD®. All differences were statistically significant according to Tukey's multiple comparisons test, except the differences of bioT12 fluorescence in brain vs striated muscle (SM) and lung vs heart.

FIG. 14. Mice deficient in GPBP are refractory to cancer implantation and spreading. Ten thousand LLC cells were inoculated subcutaneously into the right rear flank 8-week old GPBP-1^{-/-} (KO) and wild type (WT) C57BL/6 mice. Tumor growth was checked by palpation. After 28 days mice were sacrificed and lungs were analyzed to determine the presence of metastases. In graphs shown are the number of C57BL/6 mice of each genotype with and without LLC tumors (left) and the number of mice that developed or not metastases. The statistical signification (P) of the differences observed among groups was assessed by Chi² Fisher's exact test with GRAPHPAD®.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All common terms between different aspects and embodiments of the invention have the same meaning unless the context clearly dictates otherwise.

Unless clearly indicated otherwise by the context, embodiments disclosed for one aspect of the invention can be used in other aspects of the invention as well, and in combination with embodiments disclosed in other aspects of the invention.

In one aspect, the present invention provides method for inhibiting mesenchymal phenotype after epithelial-to-mesenchymal transition (EMT), or for treating an invasive tumor comprising administering to a subject in need thereof an amount effective to inhibit cell survival after EMT, or to treat an invasive tumor, of an antibody selective for GPBP, or a compound of formula:

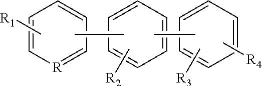

or a pharmaceutically acceptable salt thereof, wherein:
R is selected from N and CR₅;
   R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, or (heteroaryl)C₁-C₆ alkyl;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, —O(CH₂)₁₋₅—C(O)(C₁-C₆ alkoxy), (aryl)C₁-C₆ alkyl, or (heteroaryl)C₁-C₆ alkyl.

The inventors have surprisingly discovered that the compounds recited in the present claims exemplified by T12, and antibodies selective for GPBP, compromise cell viability after epithelial-to-mesenchymal transition to a much greater extent than they affect epithelial cell viability, and inhibit growth and metastasis of invasive tumors (i.e.: those having predominant mesenchymal phenotype) to a much greater extent than they effect the growth of tumors having predominant epithelial phenotype. As a result, the methods of the invention can be used, for example, to treat invasive tumors as well as disorders mediated by organ fibrosis including but not limited to chronic kidney disease, immune complex-mediated glomerulonephritis (GN) (including but not limited to IgA nephropathy, systemic lupus erythematosus (SLE) and Goodpasture disease), rheumatoid arthritis and pulmonary fibrosis (PF).

In one embodiment, the compound has the formula:

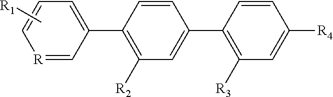

wherein:
R is selected from N and CR₅;
   R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), and —(CH₂)₁₋₅—C(O)NH₂;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), cyano, hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), or —(CH₂)₁₋₅—C(O)NH₂;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —C(O)OH, —(CH₂)₁₋₅—C(O)OH, —C(O)(C₁-C₆ alkoxy), —(CH₂)₁₋₅—C(O)(C₁-C₆ alkoxy), —C(O)NH₂, —(CH₂)₁₋₅—C(O)NH₂, —C(O)NH(C₁-C₆ alkyl), —(CH₂)₁₋₅—C(O)NH(C₁-C₆ alkyl), —C(O)N(C₁-C₆ alkyl)₂, —(CH₂)₁₋₅—C(O)N(C₁-C₆ alkyl)₂, —CH═CH—C(O)OH, or —CH═CH—C(O)(C₁-C₆ alkoxy); and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, or —O(CH₂)₁₋₅—C(O)(C₁-C₆ alkoxy).

In another embodiment:
R is selected from N and CR₅;
   R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);
R₁ is hydrogen, halogen, hydroxy, C₁-C₆ alkyl, halo(C₁-C₆ alkyl), C₁-C₆ alkoxy, or halo(C₁-C₆ alkoxy);
R₂ is C₁-C₆ alkyl, halo(C₁-C₆ alkyl), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), or (C₁-C₆ alkyl)sulfanyl(C₁-C₆ alkyl);
R₃ is C₁-C₆ alkyl, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, or —CH═CH—C(O)(C₁-C₆ alkoxy); and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, or —O(CH₂)₁₋₅—C(O)(C₁-C₆ alkoxy).

In a further embodiment, the compound has the formula:

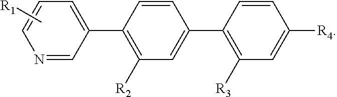

In another embodiment, the compound has the formula:

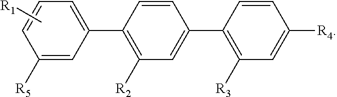

In one embodiment of any embodiment of the compounds of the invention, R₁ is hydrogen. In a further embodiment of any embodiment of the compounds of the invention, R₂ is C₁-C₆ alkyl, halo(C₁-C₆ alkyl), hydroxy(C₁-C₆ alkyl), formyl(C₀-C₆ alkyl), amino(C₁-C₆ alkyl), or sulfanyl(C₁-C₆ alkyl). In one embodiment, R₂ is C₁-C₆ alkyl, halo(C₁-C₆ alkyl), or hydroxy(C₁-C₆ alkyl).

In an embodiment of any embodiment of the compounds of the invention, R₃ is C₁-C₆ alkyl, —(CH₂)₁₋₅—C(O)OH, —(CH₂)₁₋₅—C(O)(C₁-C₆ alkoxy), —CH═CH—C(O)OH, or —CH═CH—C(O)(C₁-C₆ alkoxy). In one embodiment, R₃ is —(CH₂)₁₋₂—C(O)OH, or —(CH₂)₁₋₂—C(O)(C₁-C₆ alkoxy).

In a further embodiment of any embodiment of the compounds of the invention R₄ is hydroxy, halogen, C₁-C₆ alkyl, C₁-C₆ alkoxy, halo(C₁-C₆ alkoxy), or benzyloxy. In one embodiment, R₄ is hydroxy or C₁-C₆ alkoxy.

In another embodiment of any embodiment of the compounds of the invention, R₅ is C₁-C₆ alkyl, halo(C₁-C₆ alkyl), C₁-C₆ alkoxy, or halo(C₁-C₆ alkoxy).

In a further embodiment, R₅, if present, is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);
R₁ is hydrogen, halogen, hydroxy, C₁-C₆ alkyl, halo(C₁-C₆ alkyl), C₁-C₆ alkoxy, or halo(C₁-C₆ alkoxy);
R₂ is C₁-C₆ alkyl, halo(C₁-C₆ alkyl), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), or (C₁-C₀ alkyl)thio(C₁-C₆ alkyl);
R₃ is —(CH₂)₁₋₂—C(O)OH, —(CH₂)₁₋₂—C(O)(C₁-C₆ alkoxy), —(CH₂)₁₋₂—C(O)NH₂, —(CH₂)₁₋₂—C(O)NH(C₁-C₆ alkyl), —(CH₂)₁₋₂—C(O)N(C₁-C₆ alkyl)₂, —CH═CH—C(O)OH, —CH═CH—C(O)(C₁-C₆ alkoxy); and
R₄ is hydroxy, C₁-C₆ alkoxy, halo(C₁-C₆ alkoxy), or benzyloxy.

In one embodiment, R₁ is hydrogen;
R₂ is C₁-C₆ alkyl, halo(C₁-C₆ alkyl), hydroxy(C₁-C₆ alkyl), or formyl(C₁-C₆ alkyl);
R₃ is —(CH₂)₁₋₂—C(O)OH, —(CH₂)₁₋₂—C(O)(C₁-C₆ alkoxy), or —(CH₂)₁₋₂—C(O)NH₂;
R₄ is hydroxy or C₁-C₆ alkoxy; and
R₅, if present, is C₁-C₆ alkyl, halo(C₁-C₆ alkyl), C₁-C₆ alkoxy, or halo(C₁-C₆ alkoxy).

In another embodiment, R, if present, is selected from N and CR₅;
R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);
R₁ is hydrogen;
R₂ is C₁-C₆ alkyl;
R₃ is —(CH₂)₁₋₂—C(O)OH; and
R₄ is C₁-C₆ alkoxy.

In a further embodiment, R, if present, is selected from N and CR₅;
R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);
R₁ is hydrogen;
R₂ is methyl;
R₃ is —(CH₂)₂—C(O)OH; and
R₄ is methoxy.

In various further embodiments, the compound is selected from the group consisting of:
ethyl (E)-3-[4″-(benzyloxy)-2′-formyl-3-methyl-(1,1′;4′,1″)terphenyl-2″-yl]acrylate;
ethyl 3-[4″-hydroxy-2′-(hydroxymethyl)-3-methyl-(1,1′;4′,1″)terphenyl-2″-yl]propionate;
3-[4″-hydroxy-2′-(hydroxymethyl)-3-methyl-(1,1′;4′,1″)terphenyl-2″-yl]propionic acid;
ethyl 3-[2′-(fluoromethyl)-4″-hydroxy-3-methyl-(1,1′;4′,1″)terphenyl-2″-yl]propionate;
ethyl 3-[2′-(hydroxymethyl)-4″-metoxy-3-methyl-(1,1′;4′,1″)terphenyl-2″-yl]propionate;
3-[4″-hydroxy-2′-(hydroxymethyl)-3-(trifluoromethyl)-(1,1′;4′,1″)terphenyl-2″-yl]propionic acid;
3-[4-hydroxy-3′-(hydroxymethyl)-4′-(pyridin-3-yl)biphenyl-2-yl]propionic acid;
3-[4″-hydroxy-2″-isopropyl-3-methyl-(1,1′;4′,1″)terphenyl-2′-yl]propionic acid;
(E)-ethyl 3-[4″-(benzyloxy)-2′-formyl-3-(trifluoromethyl)-(1,1′;4′,1″)terphenyl-2″-yl]acrylate;
(E)-ethyl 3-[4-(benzyloxy)-3′-formyl-4′-(pyridin-3-yl)biphenyl-2-yl]acrylate;
ethyl 3-[4″-hydroxy-2′,3-dimethyl-(1,1′;4′,1″)terphenyl-2″-yl]propionate;
ethyl 3-[4″-hydroxy-2′-methyl-3-(trifluoromethyl)-(1,1′;4′,1″)terphenyl-2″-yl]propionate;
3-[4″-hydroxy-2′,3-dimethyl-(1,1′;4′,1″)terphenyl-2″-yl]propionic acid;
3-[4″-hydroxy-2′-methyl-3-(trifluoromethyl)-(1,1′;4′,1″)terphenyl-2″-yl]propionic acid;
ethyl 3-[4″-hydroxy-2′-(hydroxymethyl)-3-(trifluoromethyl)-(1,1′;4′,1″)terphenyl-2″-yl]propionate;
ethyl 3-[4-hydroxy-3′-(hydroxymethyl)-4′-(pyridin-3-yl)biphenyl-2-yl]propionate;
ethyl 3-[2′-(fluoromethyl)-4″-hydroxy-3-(trifluoromethyl)-(1,1′;4′,1″)terphenyl-2″-yl]propionate;
ethyl 3-[3′-(fluoromethyl)-4-hydroxy-4′-(pyridin-3-yl)biphenyl-2-yl]propionate;
3-[2′-(fluoromethyl)-4″-hydroxy-3-methyl-(1,1′;4′,1″)terphenyl-2″-yl]propionic acid;
3-[2′-(fluoromethyl)-4″-hydroxy-3-(trifluoromethyl)-(1,1′;4′,1″)terphenyl-2″-yl]propionic acid;
3-[3′-(fluoromethyl)-4-hydroxy-4′-(pyridin-3-yl)biphenyl-2-yl]propionic acid;
ethyl (E)-3-[4″-(benzyloxy)-2′-(difluoromethyl)-3-methyl-(1,1′;4′,1″)terphenyl-2″-yl]acrylate;
ethyl (E)-3-[4″-(benzyloxy)-2′-(difluoromethyl)-3-(trifluoromethyl)-(1,1′;4′,1″)terphenyl-2″-yl]acrylate;
(E)-ethyl 3-[4-(benzyloxy)-3′-(difluoromethyl)-4′-(pyridin-3-yl)biphenyl-2-yl]acrylate;
ethyl 3-[2′-(difluoromethyl)-4″-hydroxy-3-methyl-(1,1′;4′,1″)terphenyl-2″-yl]propionate;
ethyl 3-[2′-(difluoromethyl)-4″-hydroxy-3-(trifluoromethyl)-(1,1′;4′,1″)terphenyl-2″-yl]propionate;
ethyl 3-[3′-(difluoromethyl)-4-hydroxy-4′-(pyridin-3-yl)biphenyl-2-yl]propionate;
3-[2′-(difluoromethyl)-4″-hydroxy-3-methyl-(1,1′;4′,1″)terphenyl-2″-yl]propionic acid;
3-[2′-(difluoromethyl)-4″-hydroxy-3-(trifluoromethyl)-(1,1′;4′,1″)terphenyl-2″-yl]propionic acid;
3-[3′-(difluoromethyl)-4-hydroxy-4′-(pyridin-3-yl)biphenyl-2-yl]propionic acid;
ethyl 3-[4″-methoxy-3,2′-dimethyl-(1,1′;4′,1″)terphenyl-2″-yl]propionate;
ethyl 3-[2′-(fluoromethyl)-4″-metoxy-3-methyl-(1,1′;4′,1″)terphenyl-2″-yl]propionate;
ethyl 3-[2′-(difluoromethyl)-4″-metoxy-3-methyl-(1,1′;4′,1″)terphenyl-2″-yl]propionate;
ethyl 3-[2′-(hydroxymethyl)-4″-metoxy-3-(trifluoromethyl)-(1,1′;4′,1″)terphenyl-2″-yl]propionate;
ethyl 3-[2′-methyl-4″-metoxy-3-(trifluoromethyl)-(1,1′;4′,1″)terphenyl-2″-yl]propionate;
ethyl 3-[2′-(fluoromethyl)-4″-metoxy-3-(trifluoromethyl)-(1,1′;4′,1″)terphenyl-2″-yl]propionate;
ethyl 3-[2′-(difluoromethyl)-4″-metoxy-3-(trifluoromethyl)-(1,1′;4′,1″)terphenyl-2″-yl]propionate;
ethyl 3-[3′-(hydroxymethyl)-4-metoxy-4′-(pyridin-3-yl)biphenyl-2-yl]propionate;
ethyl 3-[4-methoxy-3′-methyl-4′-(pyridin-3-yl)biphenyl-2-yl]propionate;
ethyl 3-[3′-(fluoromethyl)-4-metoxy-4′-(pyridin-3-yl)biphenyl-2-yl]propionate;
ethyl 3-[3′-(difluoromethyl)-4-metoxy-4′-(pyridin-3-yl)biphenyl-2-yl]propionate;
3-[2′-(hydroxymethyl)-4″-methoxy-3-methyl-(1,1′;4′,1″)terphenyl-2″-yl]propionic acid;
3-[4″-methoxy-3,2′-dimethyl-(1,1′;4′,1″)terphenyl-2″-yl]propionic acid;
3-[2′-(fluoromethyl)-4″-methoxy-3-methyl-(1,1′;4′,1″)terphenyl-2″-yl]propionic acid;
3-[2′-(difluoromethyl)-4″-methoxy-3-methyl-(1,1′;4′,1″)terphenyl-2″-yl]propionic acid;
3-[2′-(hydroxymethyl)-4″-methoxy-3-(trifluoromethyl)-(1,1′;4′,1″)terphenyl-2″-yl]propionic acid;
3-[2′-methyl-4″-methoxy-3-(trifluoromethyl)-(1,1′;4′,1″)terphenyl-2″-yl]propionic acid;
3-[2′-(fluoromethyl)-4″-methoxy-3-(trifluoromethyl)-(1,1′;4′,1″)terphenyl-2″-yl]propionic acid;
3-[2′-(difluoromethyl)-4″-methoxy-3-(trifluoromethyl)-(1,1′;4′,1″)terphenyl-2″-yl]propionic acid;
3-[3′-(hydroxymethyl)-4-methoxy-4′-(pyridin-3-yl)-biphenyl-2-yl]propionic acid;
3-[4-methoxy-3′-methyl-4′-(pyridin-3-yl)biphenyl-2-yl]propionic acid;
3-[3′-(fluoromethyl)-4-methoxy-4′-(pyridin-3-yl)-biphenyl-2-yl]propionic acid;
3-[3′-(difluoromethyl)-4-methoxy-4′-(pyridin-3-yl)-biphenyl-2-yl]propionic acid;
ethyl 3-[3,2′-dimethyl-4″-propoxy-(1,1′;4′,1″)terphenyl-2″-yl]propionate;
ethyl 3-[4″-(ethoxycarbonylmethoxy)-3,2′-dimethyl-(1,1′;4′,1″)terphenyl-2″-yl]propionate;
ethyl 3-[2′-methyl-4″-propoxy-3-(trifluoromethyl)-(1,1′;4′,1″)terphenyl-2″-yl]propionate;
3-[3,2′-dimethyl-4″-propoxy-(1,1′;4′,1″)terphenyl-2″-yl]propionic acid;
3-[4″-(carboxymethoxy)-3,2′-dimethyl-(1,1′;4′,1″)terphenyl-2″-yl]propionic acid;
3-[2′-methyl-4″-propoxy-3-(trifluoromethyl)-(1,1′;4′,1″)terphenyl-2″-yl]propionic acid;
ethyl 3-[3′-formyl-4-metoxy-4′-(pyridin-3-yl)biphenyl-2-yl]propionate;
ethyl 3-[4,4″-dimethoxy-3,2′-dimethyl-(1,1′;4′,1″)terphenyl-2″-yl]propionate;
3-[4,4″-dimethoxy-3,2′-dimethyl-(1,1′;4′,1″)terphenyl-2″-yl]propionic acid;
ethyl (E)-3-[4″-(benzyloxy)-3-formyl-2″-isopropyl-(1,1′;4′,1″)terphenyl-2′-yl]acrylate;
ethyl 3-[4″-hydroxy-2″-isopropyl-3-methyl-(1,1′;4′,1″)terphenyl-2′-yl]propionate;
3-[3-chloro-2′-methyl-4,4″-dimethoxy-(1,1′;4′,1″)terphenyl-2″-yl]propionic acid;
   or a pharmaceutically acceptable salt thereof.

In one specific embodiment, the compound is 3-[4″-methoxy-3,2′-dimethyl-(1,1′;4′,1″)terphenyl-2″-yl] propionic acid, or a pharmaceutically acceptable salt thereof. This compound is also referred to as T12, having the structure below (compound 22b in WO2011/054530):

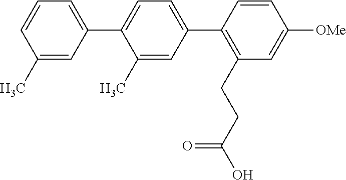 [SMILES: COc1ccc(-c2ccc(-c3cccc(C)c3)c(C)c2)c(CCC(=O)O)c1]

Methods for making the compounds for use in the present invention are disclosed in WO2011/054530 and U.S. Pat. No. 9,066,938, incorporated by reference herein in its entirety.

In another embodiment, the methods comprise administering to the subject an antibody selective for GPBP. Any suitable GPBP inhibitor may be used in the methods of the invention. In one embodiment, the GPBP inhibitor comprises an anti-GPBP antibody, such as a monoclonal or polyclonal antibody. As used herein, “anti-GPBP antibody” means that the antibodies bind to all or individual GPBP isoforms. In a preferred embodiment that can be combined with any other embodiment, the antibody is a monoclonal antibody, such as a humanized monoclonal antibody. The term antibody as used herein is intended to include antibody fragments thereof which are selectively reactive with the polypeptides of the invention, or fragments thereof. Antibodies can be fragmented using conventional techniques or synthesized through genetic engineering using recombinant DNA, and the fragments screened for utility in the same manner as described above for whole antibodies. For example, F(ab′)2 fragments can be generated by treating antibody with pepsin. The resulting F(ab′)2 fragment can be treated with papain to produce Fab fragments. Examples of monoclonal antibody fragments include (i) a Fab fragment, a monovalent fragment consisting essentially of the VL, VH, CL and CH1 domains; (ii) F(ab)2 and F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, such as scFV, tandem di-scFV, diabodies, tri(a)bodies, etc. (v) a dAb fragment (Ward et al., (1989) Nature 341:544-546), which consists essentially of a VH domain; and (vi) one or more isolated CDRs or a functional paratope. Exemplary antibodies are disclosed, for example, in WO 2010/009856 and U.S. Pat. No. 7,935,492. In one embodiment, the antibodies recognize native 77 kD GPBP, including but not limited to those antibodies disclosed in WO 2010/009856 and U.S. Pat. No. 7,935,492, which provides teachings for those of skill in the art to generate antibodies to native 77 kD GPBP. As used herein, “antibodies to native 77 kD GPBP” means that the antibodies bind to native 77 kD GPBP, and does not require that they not bind to other GPBP species. In one embodiment, the antibodies are specific for 77 kD GPBP. In a further preferred embodiment that can be combined with any other embodiment, the antibody is a monoclonal antibody, such as a humanized monoclonal antibody.

Throughout EMT epithelial cells undergo trans-differentiation towards a phenotype with an enhanced migratory capacity and invasiveness, high resistance to apoptosis and an outstanding capacity to synthesize extracellular matrix (see for review Kalluri et al., 2009, J. Clin. Invest. 119:1420-8). Whereas different EMTs have been recognized in embryo implantation and development (type 1); tissue repair and organ fibrosis (type 2); or cancer malignancy and metastasis formation (type 3), the general consensus is that common molecular mechanism must exist among them. Thus, in embodiments of the invention for inhibiting mesenchymal phenotype after EMT, the subject may be one that has or is suspected of having any disorder characterized by EMT, including but not limited to chronic kidney disease, immune-complex mediated glomerulonephritis, organ fibrosis, pulmonary fibrosis, rheumatoid arthritis, and an invasive tumor. As will be understood by those of skill in the art, not all cells undergo EMT at once. EMT transition occurs in mosaic fashion and may occur more prominently at the borders of the affected tissue, such as a tumor.

In embodiments where the methods are for treating an invasive tumor, the invasive tumor may be an invasive carcinoma, including but not limited to invasive breast tumors and invasive lung tumors. In a further embodiment, treating the invasive tumor reduces tumor metastases in the subject.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

Inhibiting mesenchymal phenotype after EMT may comprise promoting cell death in such cells and/or promoting transition of such cells back to an epithelial phenotype.

Dosage levels of the order of from about 0.01 mg to about 50 mg per kilogram of body weight per day, and more preferably between 0.1 mg to about 50 mg per kilogram of body weight per day, are useful in the treatment of the above-indicated conditions. The amount of active ingredient that may be combined with the carrier materials to produce a single dosage form will vary depending upon the host treated and the particular mode of administration. Dosage unit forms will generally contain between from about 1 mg to about 500 mg of an active ingredient.

Compounds, antibodies, or pharmaceutical compositions containing the compounds or antibodies described herein are administered to an individual in need thereof. In a preferred embodiment, the subject is a mammal; in a more preferred embodiment, the subject is a human. In therapeutic applications, compositions are administered in an amount sufficient to carry out the methods of the invention. Amounts effective for these uses depend on factors including, but not limited to, the nature of the compound (specific activity, etc.), the route of administration, the stage and severity of the disorder, the weight and general state of health of the subject, and the judgment of the prescribing physician. The active compounds are effective over a wide dosage range. However, it will be understood that the amount of the compound actually administered will be determined by a physician, in the light of the above relevant circumstances. Therefore, the above dosage ranges are not intended to limit the scope of the invention in any way.

E-cadherin expression supports cell-cell attachment in epithelial phenotype and vimentin expression renders cells prone to cell-cell detachment and migration in mesenchymal phenotype. Collagen IV is a primary component of the extracellular matrix that interacts with cancer stem cells (CSCs) forming a protective shield against conventional anti-tumor therapies (Ye J et al., 2014, Tumour Biol. 35, 3945-51; Su C et al.,2007, Cancer Invest. 2, 542-9). In one embodiments, the subject to be treated is identified as having an issue related to EMT based on an altered expression of cell markers in a relevant tissue sample compared to a control tissue sample, wherein the altered expression is indicative of an epithelial-to-mesenchymal phenotype transition. For example, the cell markers may include but are not limited to one or more of vimentin, E-cadherin, collagens I and IV, MMP-9, CCL2/MCP-1, α5 (IV) chain, (α5 (IV))₃ protomer, and Goodpasture antigen binding protein (GPBP). These markers are consistently altered (i.e.: increased (such as vimentin and collagen I and IV, α5 (IV), (α5 (IV))₃, MMP-9, CCL2/MCP-1, and GPBP) or decreased (such as E-cadherin)) after EMT. Any suitable technique for detecting marker levels (mRNA and/or protein) can be used, including but not limited to immunohistochemistry and in situ hybridization on tissue biopsies (such as a tumor biopsy). In a specific embodiment, the subject has an increase in vimentin expression and a decrease in E-cadherin expression in a relevant tissue sample compared to an epithelial cell control.

Such “increase” can be any amount of increase relative to control (such as control sample from a normal subject, or previously determined “normal” levels of the marker in a control population), for example, 5%, 10%, 15%, 20%, 25%, 50%, 75%, 100%, or greater.

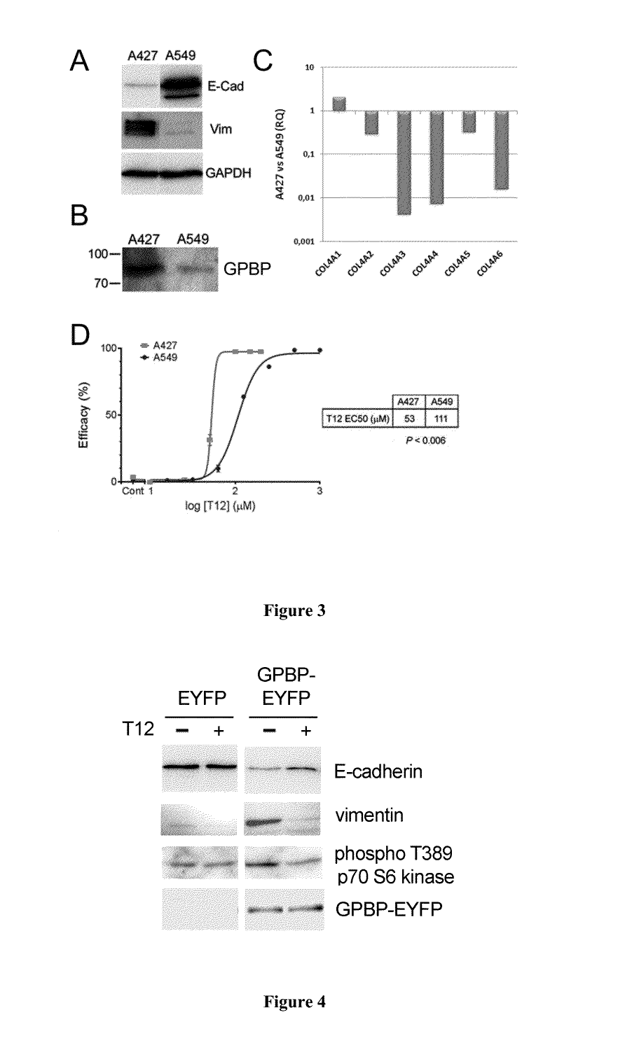

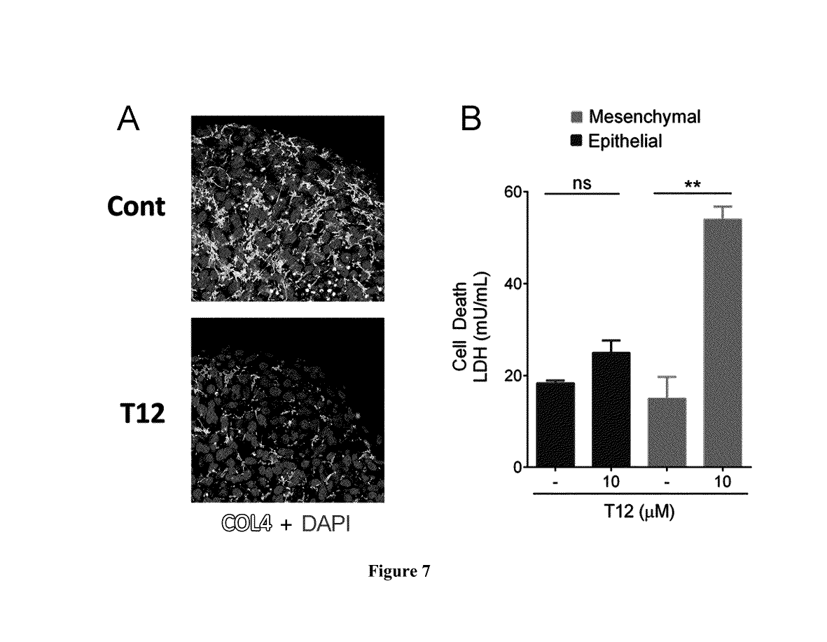

As described in the examples that follow, the authors have found that mesenchymal phenotype expresses along with the classical collagen IV made of α1 α2 chains a previously unrecognized collagen IV made of α5 chain. There is evidence that the compounds for use in the invention, exemplified by T12, reduce the expression of the α1 α2α5 chains. This provides compelling evidence that a previously unrecognized collagen IV network (made up of the α1 α2α5 chains) supports mesenchymal phenotype, and is different in composition than the typical collagen IV networks which supports epithelial phenotypes (α1 α2, α3α4α5 and α5α6). This is also supported by analysis of a macrophage-based leukemia cell line (Raw 264.7) which is of mesenchymal origin, and was found to unexpectedly express only significant levels of α5(IV) collagen chain. Thus, the new mesenchymal collagen IV made of the α5 chain can be used to identify mesenchymal tumor cells: detecting α1,α2,α5 chains in a tumor in absence of significant expression of α3,α4,α6 chains will be indicative of EMT in a carcinoma; detecting α5 and no significant levels of α1, α2, α3,α4, α6 in a tumor will be indicative of sarcoma. Finally, inhibition of the mesenchymal collagen IV made of the α1,α2, α5 chains using the inhibitors described herein results in either death of mesenchymal or tumor cells, or reversion of the cell phenotype to epithelial (See, for examples, FIGS. 3 and 7).

Thus, in one embodiment the methods comprise identifying a subject to be treated as one with an increased expression of α5(IV) chain, and (α5(IV))₃ protomer, and/or with an increased expression of α1,α2(IV) chains and collagen (α1)₂α2 protomer, in a relevant tissue sample compared to a control tissue sample, wherein the increase expression is indicative of an epithelial-to-mesenchymal phenotype transition and/or an invasive tumor phenotype.

In a further embodiment, the subject to be treated is one with a reduced expression of α3, α4, α6(IV) chains or a reduced expression of α1, α2, α3, α4, α6(IV) chains in the relevant tissue (such as the tumor). Such “reduced expression” can be any amount of decrease relative to control, for example, 5%, 10%, 15%, 20%, 25%, 50%, 75%, 100%, or undetectable expression.

Similarly, a course of treatment can be monitored by determining expression of α5(IV) chain and (α5(IV))₃ protomer, and/or α1,α2(IV) chains and/or (α1)₂α2(IV) protomer in a relevant tissue sample compared to a control tissue sample (in this case, for example, a sample from the subject prior to treatment or from earlier during the course of treatment), wherein a decreased expression indicates an effective course of treatment.

The compounds or antibodies for administration include pharmaceutically acceptable salts, esters, amides, and prodrugs thereof, including but not limited to carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.)

Examples of pharmaceutically acceptable, non-toxic esters of the compounds include C₁-C₆ alkyl esters, wherein the alkyl group is a straight or branched, substituted or unsubstituted, C₅-C₇ cycloalkyl esters, as well as arylalkyl esters such as benzyl and triphenylmethyl. C₁-C₄ alkyl esters are preferred, such as methyl, ethyl, 2,2,2-trichloroethyl, and tert-butyl. Esters of the compounds of the present invention may be prepared according to conventional methods. Examples of pharmaceutically acceptable, non-toxic amides of the compounds include amides derived from ammonia, primary C₁-C₆ alkyl amines and secondary C₁-C₆ dialkyl amines, wherein the alkyl groups are straight or branched. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

The term “prodrug” refers to compounds that are rapidly transformed in vivo to yield the parent compound of the above formulae, for example, by hydrolysis in blood. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

For administration, the compounds or antibodies are ordinarily combined with one or more adjuvants appropriate for the indicated route of administration. The compounds or antibodies may be mixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds or antibodies may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The compounds or antibodies can be administered as the sole active therapeutic agent, or they can be used in combination with one or more other compounds useful for carrying out the methods of the invention. When administered as a combination, the therapeutic agents can be formulated as separate compositions that are given at the same time or different times, or the therapeutic agents can be given as a single composition

The compounds or antibodies may be made up in a solid form (including granules, powders or suppositories) or in a liquid form (e.g., solutions, suspensions, or emulsions). The compounds or antibodies may be applied in a variety of solutions and may be subjected to conventional pharmaceutical operations such as sterilization and/or may contain conventional adjuvants, such as preservatives, stabilizers, wetting agents, emulsifiers, buffers etc.

The compounds or antibodies may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of the invention and a pharmaceutically acceptable carrier. One or more compounds or antibodies of the invention may be present in association with one or more non-toxic pharmaceutically acceptable carriers and/or diluents and/or adjuvants, and if desired other active ingredients. The pharmaceutical compositions may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases, such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. In addition, fatty acids such as oleic acid find use in the preparation of injectables.

Compounds and pharmaceutical compositions of the present invention may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

In another aspect, the invention provides methods for detecting EMT in a tissue, comprising

(a) contacting a tissue in a subject with an amount effective to label the tissue of a detectably labeled compound of formula:

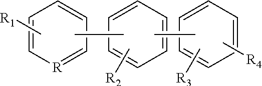

or a pharmaceutically acceptable salt thereof, wherein:
R is selected from N and CR₅;
   R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, or (heteroaryl)C₁-C₆ alkyl;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, —O(CH₂)₁₋₅—C(O)(C₁-C₆ alkoxy), (aryl)C₁-C₆ alkyl, or (heteroaryl)C₁-C₆ alkyl;
   for a time and under conditions suitable to promote binding of the detectably labeled compound to the tissue; and
   (b) detecting the detectably labeled compound bound to the tissue, thereby detecting EMT in the tissue,

As shown in the examples that follow, the inventors have discovered that detectably labeled versions of the compounds and antibodies for use in the invention, exemplified by T12, bind specifically to extracellular GPBP multimers typically present in the tumor or organs undergoing fibrosis, and thus can be used to detect EMT in a tissue.

Throughout EMT epithelial cells undergo trans-differentiation towards a phenotype with an enhanced migratory capacity and invasiveness, high resistance to apoptosis and an outstanding capacity to synthesize extracellular matrix (see for review Kalluri et al., 2009, J. Clin. Invest. 119:1420-8). Whereas different EMTs have been recognized in embryo implantation and development (type 1); tissue repair and organ fibrosis (type 2); or cancer malignancy and metastasis formation (type 3), the general consensus is that common molecular mechanism must exist among them. Thus, the tissue is selected from the group consisting of a tumor, a joint, and tissue from any organ. In one embodiment, the tissue is a kidney, and detecting EMT in the kidney indicates that the subject has chronic kidney disease or immune-complex mediated GN. In another embodiment, the tissue is tissue from any organ, and detecting EMT indicates that the subject has organ fibrosis. In a further embodiment, the tissue is a lung, and detecting EMT in the lung indicates that the subject has pulmonary fibrosis. In another embodiment, the tissue is a joint, and wherein detecting EMT indicates that the subject has rheumatoid arthritis. In a further embodiment, the tissue is a tumor, and wherein detecting EMT indicates that the subject has an invasive tumor, such as an invasive carcinoma (including but not limited to invasive breast tumors and invasive lung tumors).

The compounds for use in the methods of this aspect of the invention can be any suitable compound or antibody as disclosed in the treatment methods above. In one specific embodiment, the compound comprises T12. The compounds or antibodies can be coupled to any suitable detectable substance. The term “coupled” is used to mean that the detectable substance is physically linked to the compound or antibody. Suitable detectable substances include various enzymes, prosthetic groups, fluorescent materials, luminescent materials and radioactive materials. Examples of suitable enzymes include horseradish peroxidase, alkaline phosphatase, (3-galactosidase, or acetylcholinesterase. Examples of suitable prosthetic-group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of suitable radioactive material include ¹²⁵I, ¹³I, ³⁵S or ³H.

Contacting the tissue may be carried out in vivo (i.e.: administering the detectably labeled compounds to the subject as appropriate) or in vitro (i.e.: contacting a tissue biopsy or other tissue specimen obtained from the subject). Methods for detecting the detectably labeled compound or antibody will depend on the detectable substance; such detection techniques are well known to those of skill in the art, and exemplary such techniques are described in the examples that follow.

The subject for all methods of the invention may be any suitable subject, including a mammal or birds such as humans, dogs, cats, cattle, horses, donkeys, pigs, chickens, turkeys, sheep, and goats.

### DEFINITIONS

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, —CH₂CH(CH₂CH₃)CH₂—.

The term “alkylene” refers to a bivalent alkyl group. An “alkylene chain” is a polymethylene group, i.e., —(CH₂)ₙ—, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The term “halo” or “halogen” as used herein, means —Cl, —Br, —I or —F.

The terms “haloalkyl”, “haloalkenyl” and “haloalkoxy” refer to an alkyl, alkenyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

The present invention may be better understood with reference to the accompanying examples that are intended for purposes of illustration only and should not be construed to limit the scope of the invention.

### Examples

Here we show that a representative Q2 peptidomimetic GPBP kinase inhibitor, 3-[4″-methoxy-3,2′-dimethyl-(1,1′;4′,1″)terphenyl-2″-yl] propionic acid (T12; (WO/2014/006020)), specifically inhibits GPBP multimers that direct the assembly and formation of the collagen IV network supporting EMT and stabilization of mesenchymal drug-resistant phenotype. GPBP multimers emerge as previously unrecognized EMT effectors with relevance in pathogenesis (i.e. organ fibrosis, cancer invasiveness and chemo resistance) and T12 as a first-in-class drug candidate to treat EMT-mediated disorders. Consistently, we have developed bioT12, a biotin-labeled T12 derivative, and showed that it binds to GPBP present in tumors but not to GPBP expressed in control tissues, suggesting that multimeric GPBP aggregation specifically associated with EMT processes.

### Results and Discussion

Extracellular GPBP is Mainly Multimeric while Intracellular GPBP is Predominantly Trimeric.

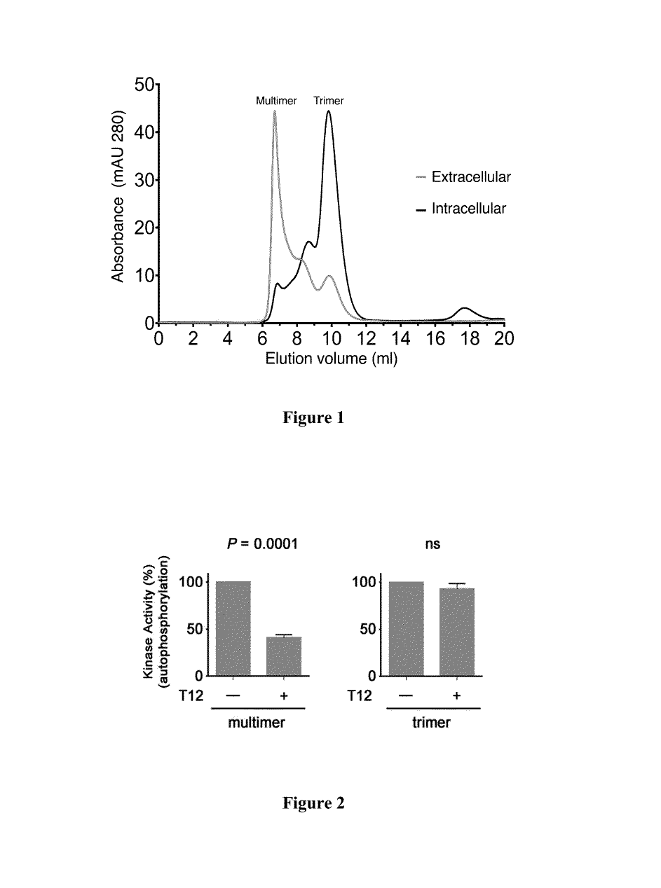

FLAG-tagged GPBP was expressed in Sf9 insect cells and secreted (extracellular) and non-secreted (intracellular) material purified by immune-affinity chromatography and further analyzed by gel filtration chromatography to assess their aggregation state. Interestingly, extracellular GPBP was found mainly as high-molecular-weight multimeric aggregates while the bulk of the intracellular GPBP material existed in a trimeric form (FIG. 1)

### T12 Inhibits the Kinase Activity of the GPBP Multimer and not the Trimer.

We used multimeric extracellular and trimeric intracellular GPBP for in vitro phosphorylation assays, and found that T12 targeted extracellular GPBP multimer but not intracellular GPBP trimers (FIG. 2), revealing that T12 is not a general GPBP kinase inhibitor but a specific inhibitor of multimeric GPBP aggregates. Consistently, when challenging intracellular GPBP multimers with T12 we found similar inhibitory effects (data not shown).

Mesenchymal Cancer A427 Cells Secrete More GPBP than Epithelial Cancer A549 Cells and are More Sensitive to T12.

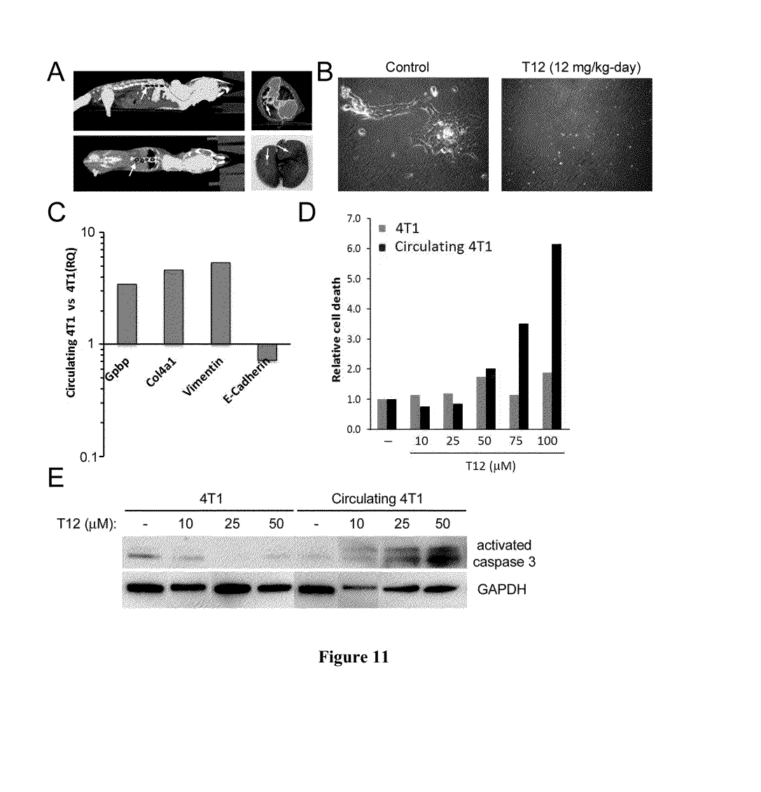

Human non-small cell lung cancer (NSCLC) displaying mesenchymal (A427) and epithelial (A549) phenotype (FIG. 3A) were cultured and GPBP immunopurified from culture media (FIG. 3B). Interestingly, A427 secreted GPBP more efficiently suggesting that enhanced GPBP expression and secretion was a mesenchymal phenotype condition. Consistently, murine Lewis Lung Cancer (LLC) and breast cancer A7C11 and 4T1 cells displaying mesenchymal phenotype expressed and secreted abundant GPBP (data not shown). Further, we investigated the expression of collagen IV and found that A549 expressed α1,α2,α3,α4,α5,α6(IV) chains whereas A427 expressed α1,α2,α5(IV) chains (FIG. 3C). Whereas the existence of (α1)₂α2, α3α4α5 and (α5)₅α6 protomers of collagen IV has been described not evidence has been reported to date for the existence of (α5)₃ protomer. To further investigate whether the expression of (α5)₃ protomers associated with mesenchymal phenotype, we analyzed collagen IV expression in epithelial cancer cells (A7C11 and 4T1) displaying mesenchymal features and in cancer cells (RAW 264.7) of primary/developmental mesenchymal origin (i.e. monocyte-macrophage leukemia). For A7C11 and 4T1 cells as for A427, we found α1,α2,α5(IV) chains to be expressed; however, for RAW 264.7 cells, we found only α5(IV) to be expressed (data not shown). All this suggesting that (α5)₃ protomer exists and its expression is a feature of mesenchymal phenotype no matter this was acquired during development or adulthood. Moreover, co-expression of (α5)₃ with (α1)₂α2 emerges as characteristic of epithelial cancer cells that underwent EMT in adulthood (type 3) whereas (α5)₃ protomer expression is characteristic of tumors emerging from primary mesenchymal cells (EMT type 1). Finally, we assessed T12 cytotoxicity and found that A427 cells were more sensitive than A549 cells to T12 (FIG. 3D), suggesting that T12 is more effective compromising viability of cancer cells upon EMT predominantly expressing α1α2α5(IV). Accordingly, further induction of mesenchymal phenotype in 4T1 cells by inoculating into mice or culturing in low adherent dishes (3D cultures), resulted in cells expressing more α1α2α5(IV) or α5(IV), respectively (data not shown) and displaying more sensitivity to T12 (FIG. 11). Consistently, T12 displayed high-toxicity towards patient-derived cisplatin-resistant mesenchymal NSCLC cells (WO/2014/006020).

### T12 Counteracts Phenotype Transition Induced by GPBP in A549 Cells.

A549 cell expressing GPBP fused to the Enhanced Yellow Fluorescent Protein (GPBP-EYFP) were generated. Cultures expressing GPBP-EYPF levels exhibited cells larger that spread more (data not shown), suggesting that cells acquired mesenchymal phenotype features. Consistently, GPBP-EYFP expressed more vimentin and displayed higher phosphorylation of p70-S6 kinase, acknowledged to translate into mTOR activation and EMT induction (Saitoh et al., 2002, J Biol Chem. 277, 20104-12; Pon et al. 2008, Cancer Res. 68, 6524-32.). In line with these findings GPBP-EYFP reduced E-cadherin expression, and T12 counteracted all these regulatory effects (FIG. 4). This suggested that multimeric GPBP induced epithelial phenotype transition. Consistently, A549 cells expressing EYFP-based intracellular GPBP counterpart (GPBP-2-EYFP) which was not expected to form multimeric aggregates (WO 00/50607) failed in regulating these biomarkers (data not shown). Collectively, our results pinpoint extracellular multimeric GPBP as a molecular mediator in EMT.

### The Viability of Mesenchymal A549 Spheroids Depends on GPBP and Collagen IV Expression and is Compromised by T12.

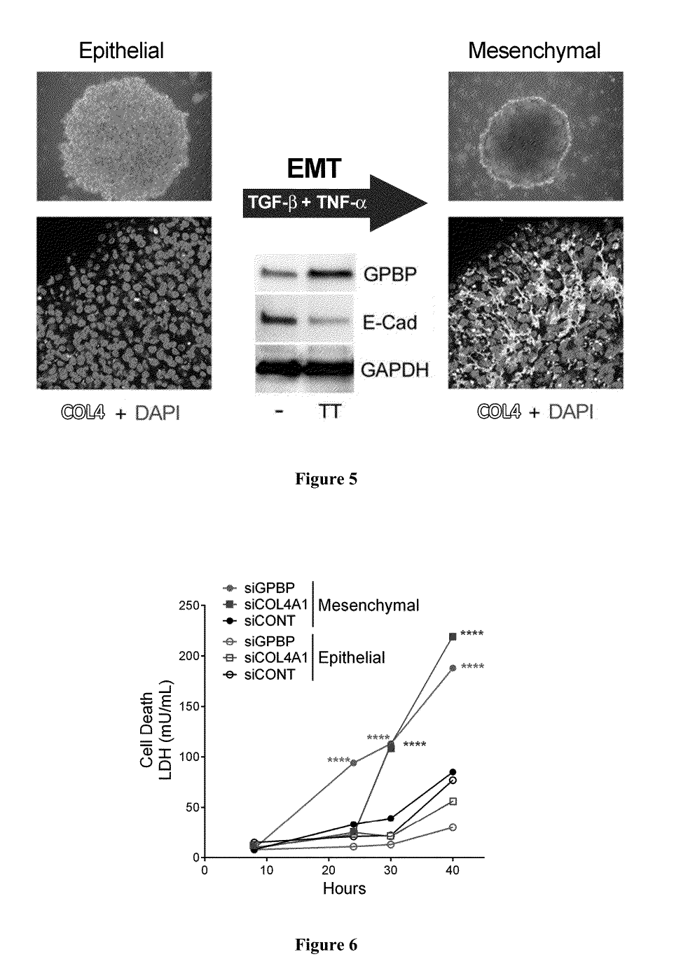

To explore the role of GPBP and its known extracellular substrate collagen IV in EMT, three-dimensional (3D) cultures, named here spheroids, which maintain stemness and mimic the growth of natural tumors, were stimulated with TNF-α and TGF-β (Kumar M et al., 2013. PLoS One. 8: e68597; Kalluri et al., 2009, J. Clin. Invest. 119:1420-8; López-Novoa et al., 2009, EMBO Mol Med 1, 303-14) and further analyzed (FIG. 5). An increase in GPBP expression and collagen IV network formation was associated with EMT induction. Subsequently, using RNA interference procedures we found that a reduction in GPBP or collagen IV expression compromised the viability of mesenchymal A549 spheroids (FIG. 6). The data suggested that GPBP multimers directed collagen IV network formation and stabilized mesenchymal phenotype. Accordingly, T12 inhibited collagen IV network formation (FIG. 7A) and reduced cell viability (FIG. 7B) of mesenchymal A549 spheroids. Moreover, T12 sharply reduced fibrillary collagen I in mesenchymal A549 spheroids and significantly reduced collagen IV expression in A7C11 and RAW264.7 (data not shown).

### T12 Abates Drug-Resistance of Mesenchymal A549 Spheroids by Inhibiting Collagen IV Network Formation.

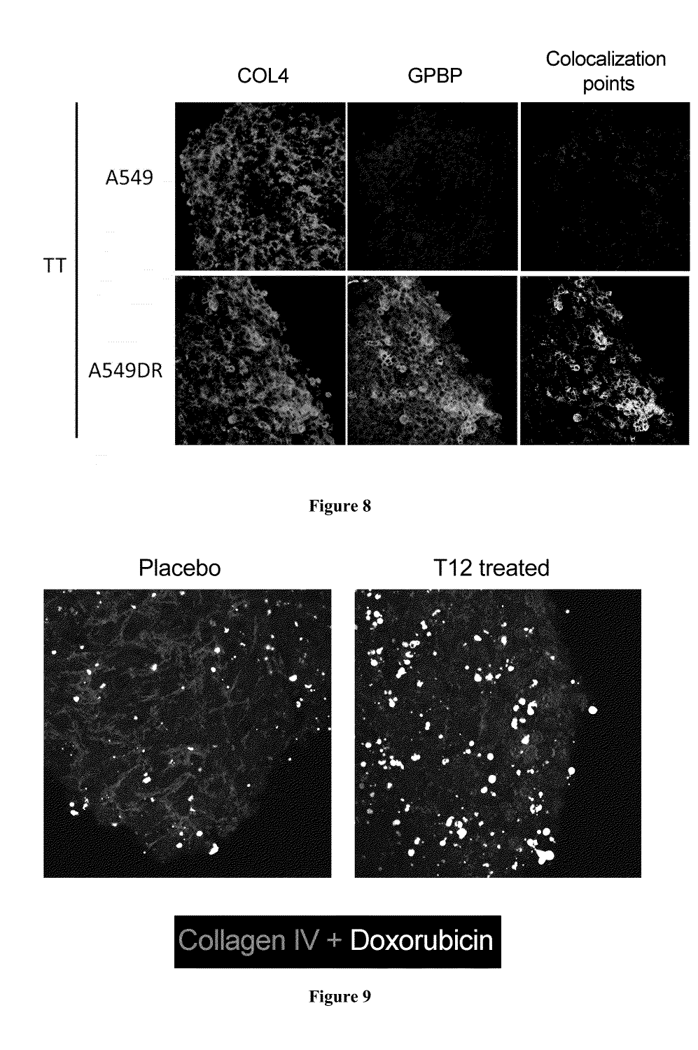

We have previously shown that T12 accumulated doxorubicin reducing viability of stem A549 cells and doxorubicin resistance associated with GPBP increased expression (WO/2014/006020). Now we have found that mesenchymal spheroids of doxorubicin-resistant A549 cells expressed increased GPBP associated with the collagen IV network (FIG. 8) and that T12 by disrupting collagen IV network and accumulating doxorubicin inside the cells sharply reduced the viability of mesenchymal A549 spheroids (FIG. 9). Collectively, these results suggest that multimeric GPBP induce the assembly of a collagen IV network that shields mesenchymal cells against chemo therapy. T12 through inhibition of kinase activity of GPBP multimers severely impairs collagen IV network formation and abates mesenchymal cell chemo resistance.

T12 is an Effective Agent Against Tumors with Mesenchymal Phenotype but Requires Sensitization by Doxorubicin to Confront Tumors with Epithelial Phenotype.

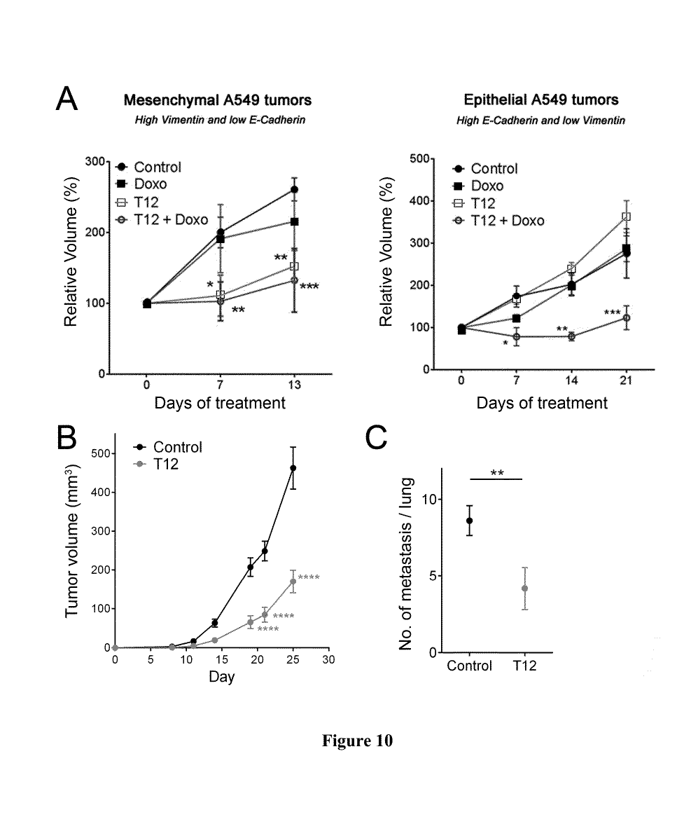

We have previously proposed that T12 is synergistic with doxorubicin reducing A549 tumor growth (WO/2014/006020). Now we have assessed the efficacy of T12 against A549 tumors displaying mesenchymal phenotype (high vimentin and low E-cadherin expression), and compared with previously reported efficacy (WO/2014/006020) against A549 tumors with epithelial phenotype (high E-cadherin and low vimentin expression) (FIG. 10A). We found that T12 was very effective slowing the growth of mesenchymal tumors in contrast with its effect on the growth of A549 epithelial tumors in which case, T12 required the synergistic action of doxorubicin to efficiently inhibit tumor growth (WO/2014/006020). The data stress the specificity of T12 as an anti-mesenchymal tumor agent. Consistently, T12 displayed anti-tumor activity and inhibited metastasis formation in murine breast cancer 4T1 model, a mouse model for mesenchymal tumors that forms abundant lung metastases because 4T1 cells are syngenic with the immunocompetent Balb/c mice and are not rejected by the mouse immune system when inoculated into the mammary pads of female mice. (FIG. 10B, C).

### T12 Targets Circulating Tumor Cells.

One of the properties of cancer cells undergoing EMT is the proneness to migrate and metastasize (Kalluri et al., 2009, J. Clin. Invest. 119:1420-8). Inoculated 4T1 cells form primary tumors that metastasize into lungs in a matter of days (FIG. 11A). Circulating 4T1 cells can be selectively cultured from blood by using culture media supplemented with 6-thioguanine. Interestingly, we found that treatment with T12 impeded the isolation of circulating 4T1 tumor cells from mice (FIG. 11B). As expected blood-isolated circulating 4T1 cells exhibited higher vimentin, Col4a1 and Gpbp expression and lower E-cadherin expression than 4T1 cells indicating that 4T1 cells had undergone EMT and acquired migratory capacity (FIG. 11C). Circulating 4T1 cells were more sensitive and underwent apoptotic cell death (caspase 3 activation) at T12 concentrations that had little effect on 4T1 cells (FIG. 11D-E). Our results further stress that mesenchymal cancer cells, including circulating cells leading to metastasis, are preferred targets of the T12 antitumor activity.

Biotinylated T12 (bioT12) Allows Specific Detection of Tumors.

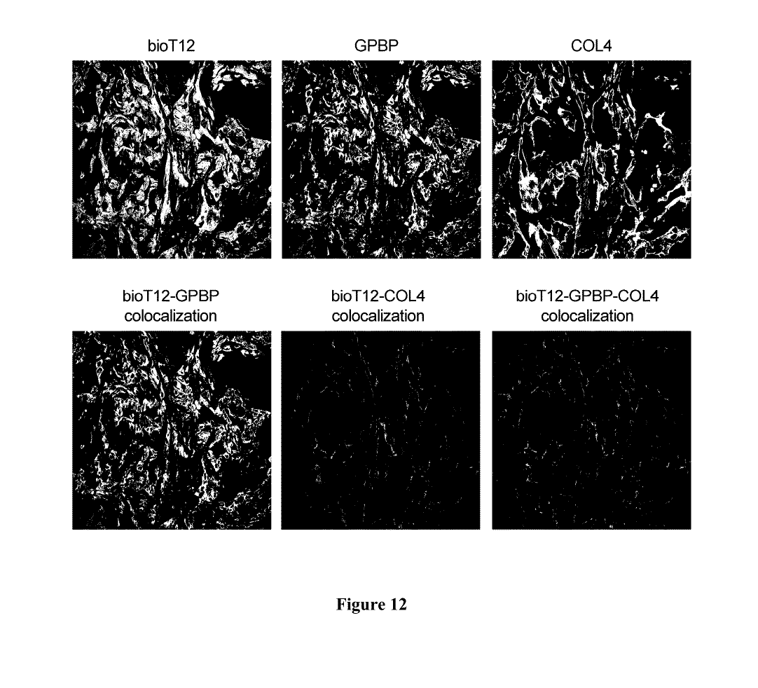

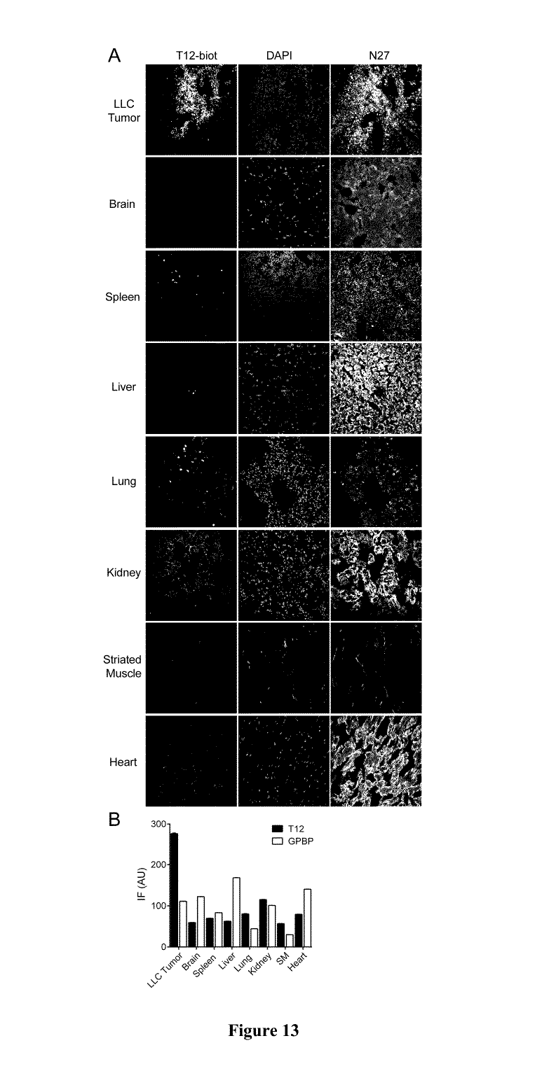

To investigate T12 binding specificity in the tumors we have generated bioT12, a derivative conjugate retaining inhibitory activity (data not shown), and used for immunostaining purposes with fluorophore-conjugated streptavidin. As expected confocal microscopy analyses of A549 tumors grown in nude mice (immune-deficient) displayed extensive co-localization of bioT12 and GPBP, revealing that T12 binds GPBP in the tumor (FIG. 12). In a more limited extent bioT12 also co-localized with collagen IV at spots where GPBP was also present, unveiling the extracellular distribution of inhibitory T12 sites from where T12 exerts his anti-tumor activity. In order to further explore T12 binding specificity we used bioT12 to stain Lewis Lung Carcinoma (LLC) tumors grown in C57BL/6 mice (immune-competent) and different tissues from control mice (FIG. 13). Intriguingly, bioT12 displayed intense and extensive staining of LLC tumor but very limited staining of control tissues despite control tissues stained significantly with GPBP-specific antibodies. Consistently, bioT12 stained human lung and breast cancer and also kidneys from patients with immune-complex mediated glomerulonephritis (IgA nephropathy) or focal segmental sclerosis undergoing tubule-interstitial fibrosis. In contrast, as for control murine tissues above, no significant staining in control tissues was observed (data not shown). Collectively, our data suggest that T12 exerts its anti-tumor activity specifically binding extracellular GPBP multimers typically present in the tumor or organs undergoing fibrosis.

### The Pro-Tumoral Activity of GPBP is Exerted, at Least in Part, at the Extracellular Compartment.

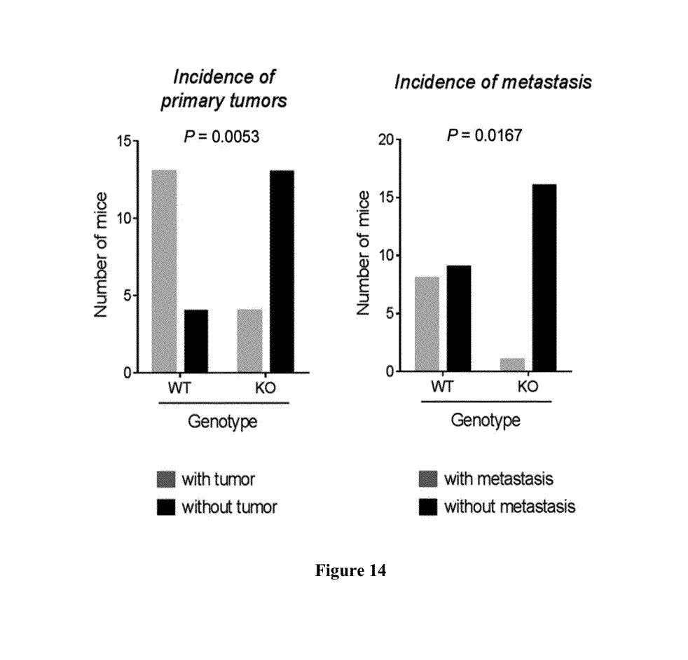

To confirm that T12 anti-tumor activity is mediated by extracellular GPBP we used GPBP-specific N26 monoclonal antibody (WO 2010/009856) to treat A549 and 4T1 cancer models. N26 yielded similar therapeutic effects than when treating those models with T12 but we found not cooperative therapeutic effects when antibody and inhibitor were combined (data not shown). This revealed that both antibody and inhibitor shared therapeutic target that must be located at the extracellular compartment accessible to the antibodies. This conclusion was further supported by demonstrating that GPBP deficient mice (GPBP-1^{-/-}) previously reported (Revert et al., 2011, J Biol Chem 286, 35030-43), displayed reduced capacity to implant primary and secondary (metastases) LLC tumors (FIG. 14).

Collectively results suggested that GPBP from the host is recruited by the tumor to form multimeric aggregates which are critical for tumor progression and dissemination.

### Experimental Procedures

### Expression and Purification of Recombinant GPBP

FLAG®-tagged GPBP was expressed using Bac-to-Bac® Baculovirus Expression System. For this purpose, FLAG®-GPBP cDNA was cloned in pFASTBAC® vector (Thermo Fisher Scientific). The resulting construct (pFASTBAC®-FLAG-GPBP) was used to transform Escherichia coli DH10BAC® bacteria (Thermo Fisher Scientific) where FLAG®-GPBP cDNA undergoes transposition into a bacmid genome. The DNA of the resulting bacmid was isolated and used to transfect Sf9 insect cells (Invitrogen). Virus particles were produced and used to infect new Sf9 cells for virus amplification that allows subsequent large scale infection and protein production. Recombinant protein expression is driven by the promoter of polyhedrin protein of virus capsid. Secreted FLAG®-GPBP was purified from culture medium of Sf9 cells 72 h after infection. Medium was centrifuged at 500×g for 10 min to pellet the cells. The supernatant was ultracentrifuged at 160,000×g (1 h, 4° C.) to pellet virus particles, and the final supernatant was filtered with 45-μm-pore-size filter and then extracted with an anti-FLAG® Affinity Gel (Sigma-Aldrich) column. The column was washed with 25 bed volumes of TBS (Tris-buffered saline) and bound FLAG®-GPBP was eluted with FLAG® peptide (0.1 mg/mL in TBS). Elutions were subjected to three cycles of ultrafiltration with Amicon Ultra Centrifugal Filters (10K) (Merck Millipore) alternated with TB S-dilutions to eliminate FLAG peptide. Finally, purified protein was quantified with Bio-Rad Protein Assay and stored at −20° C. For purification of intracellular FLAG®-GPBP, Sf9 cells were lysed at 4° C. during 30 min in TBS supplemented with 0.1% Triton X-100, 1 mM PMSF, 10 μg/mL leupeptine and 10 μg/mL benzamidine. Lysates were centrifuged at 16,000×g (1 h, 4° C.) and supernatants filtered with 45-μm-pore-size filter and purified with anti-FLAG® Affinity Gel as above indicated.

### Gel Filtration Chromatography

Gel filtration studies were performed with a SUPERDEX® 200 column (GE Healthcare) and an ÄKTA® purifier (GE Healthcare). Typically, from 150 to 250 μg of either secreted extracellular or intracellular purified FLAG®-GPBP were loaded onto the column and chromatography was performed using TBS as mobile phase. Five hundred-μl fractions were collected with a Frac-920 collector (GE Healthcare) and stored at −80° C. until use.

### In Vitro Phosphorylation Assays

Typically, about 270 ng of purified FLAG®-GPBP protein, either extracellular multimer or intracellular trimer, were incubated in presence or absence of T12 (50) in a kinase assay buffer containing 25 mM disodium β-glycerophosphate, 8 mM MgCl₂, 0.5 mM EDTA, 0.5 mM EGTA, 1 mM DTT, 5 mM MnCl₂ during 10 min at 37° C. Then γ[³²P]ATP was added to a final concentration of 0.132 μM and reactions were allowed to proceed during 15 min at 37° C. Then reactions were stopped with SDS-PAGE loading buffer and heat (95° C., 3 min) and analyzed by SDS-PAGE, electro-transference to PVDF membrane (Merck Millipore) and autoradiography. Proteins were visualized with anti-FLAG monoclonal antibodies and chemo-luminescence (ECL, GE Healthcare).

### Cell Culture

Insect 519 cells were cultured in Sf-900™ II SFM medium (Thermo Fisher Scientific) supplemented with 0.5% Pluronic® F-68 (Sigma-Aldrich).

Mouse 4T1 breast cancer cells were cultured in RPMI 1640 medium (Lonza) supplemented with 10% fetal bovine serum (FBS). Mouse Lewis Lung Carcinoma (LLC) cells were cultured in High-glucose (4.5 g/L) DMEM (Lonza) supplemented with 10% FBS. To isolate and culture circulating 4T1 cells, blood from 4T1-innoculated Balb/c mice was collected at the end of the assay, erythrocytes lysed with sterile Red Blood Cell Lysis Buffer (GIBCO, A10492-01) by repeated (3 times) centrifugation (500×g, 5 min). Final cellular pellet was washed, dispersed and cultured with DMEM (Lonza) supplemented with 10% FBS supplemented with 60 μM 6-thioguanine.

Human A427 and A549 cell lines were cultured in DMEM-F12 (Lonza) containing 15 mM Hepes and 2.5 mM L-Gln and supplemented with 10% FBS.

All media were supplemented with 100 U/mL penicillin and 100 μg/mL streptomycin.

### Cell Viability Assays

Cell viability assays were performed either with ALAMARBLUE® reagent (Thermo Fisher Scientific) or by measuring LDH activity in culture media.

For IC50 determination of T12, cells were seeded on 96-well culture plates (2,500 cells/well) and allowed to settle during 4 hours. Then cells were treated with individual compounds at several concentrations ranging from 0 to 200 μM during 36 h. Subsequently, ALAMARBLUE® reagent was added to wells and incubation maintained for 3 additional hours. Fluorescence was measured using 560EX nm/590EM nm filter settings with a SPECTRAMAX® GeminiXPS plate reader (Molecular Devices). Blank wells containing media were used to determine background fluorescence. Data processing and IC50 calculations were performed with SOFMAX® Pro software (Molecular Devices)

For some purposes, culture media were cleared by centrifugation (500×g, 10 min, room temperature) and LDH activity in supernatants determined with Lactate Dehydrogenase Activity Assay Kit (Sigma-Aldrich).

### Production of A549 Cell Lines Expressing GPBP-EYFP Fusion Protein and EYFP

To generate cells expressing GPBP fused to Enhanced Yellow Fluorescent Protein (GPBP-EYFP) or EYFP, A549 cells were transfected either with pEYFP-N1-GPBP construct or with pEYFP-N1 vector (Clontech), expressing GPBP-EYFP and EYFP, respectively. Transfected cells were selected with 400 mg/L of geniticin and clones were isolated with a High SPEED CELL SORTER MOFLO® (Beckman-Coulter) and further cultured. Recombinant protein expression was assessed by immunofluorescence microscopy and by Western blot with anti-GPBP N27 mouse monoclonal antibodies.

### Production of A549 Cells Resistant to Doxorubicin

A549 cells were cultured in presence of doxorubicin (1 μM) and medium replaced every 2 days to remove dead cells and debris. After several weeks of culture in doxorubicin-containing medium, the increase of IC50 for doxorubicin was determined to confirm the acquired resistance of surviving cells. Doxorubicin-resistant A549 cells (A549DR) were bigger and divided more slowly than original A549 cells, and were used for 3-dimensional spheroid culture.

### Three Dimensional Spheroid Cultures and EMT

Three-dimensional spheroid cultures of A549 cells were obtained using a hanging droplet method (Kelm et al., 2003. Biotechnol. Bioeng. 83: 173-180.; Kumar et al., 2013. PLoS One. 8: e68597). Briefly, cells were grown to approximately 80% confluence on adherent tissue-culture flasks. Then cells were trypsinized, dispersed in DMEM/10% FBS, and counted using an automated cell counter (MOXI® Z, Orflo). The cell suspension was diluted to a 10⁶ cells/mL-concentration, and 25 μl of the cell suspension were pipetted onto the underside of a sterile 10-cm tissue culture plate lid. Each lid was loaded with approximately 55 droplets. After loading, the lid was placed onto a tissue culture plate containing 6 mL of sterile PBS (phosphate-buffered saline) and incubated for 48 hours to facilitate spheroid formation. The freshly formed spheroids were then transferred into E-well ultra-low binding plates (Nunclon Sphera, Thermo Scientific) to prevent cell attachment to the dish bottom, and were cultured in 2 mL per well of DMEM/2% FBS. Each suspension plate typically held up to 55 spheroids. After transfer, spheroids were treated twice with 10 ng/ml of TNF-α and 2 ng/ml of TGF-β (Invitrogen) for 48 hours. Where indicated, doxorubicin resistant A549 (A549DR) cells were similarly cultured.

Three dimensional cultures of A427 cells are obtained growing cells in ultra-low binding plates with DMEM/10% SBF.

### Immunoprecipitation

For immunoprecipitation purposes, anti-GPBP mouse monoclonal antibodies N26 (Fibrostatin, SL) were conjugated to Cyanogen bromide-activated-Sepharose® 4B beads (Sigma) following manufacturer's instructions. Media from A427 and A549 cell cultures (25 mL) were immunoprecipitated with 100 μl of slurry (50:50) of sepharose-conjugated N26 antibodies overnight with gentle rocking at 4° C. Beads were recovered by centrifugation (500×g, 10 min, 4° C.) and six-times washed alternating TBST (TBS with 0.05% Tween 20) and TBS (1 mL per wash). Then beads were eluted with five bed volumes of 0.1 M Gly-HCl pH 2.7, elutions were pooled and solution buffer exchanged by repeated cycles of dilution with PBS and concentration using Amicon® Ultra Centrifugal filters 10 K (Merck Millipore). Purified materials were stored at −80° C. until use.

### Mouse Xenograft Studies

For some assays, 3×10⁶ A549 cells were suspended in 150 μl of culture medium, mixed with 150 μl of Matrigel® (Corning) and subcutaneously injected into the right flank of 8-week-old athymic NMiti-Foxn1^{nu}/Foxn1^{nu} male mice (Janvier). Tumor's size measurements were performed with a digital caliper and volumes calculated with the formula Volume=(Length×Width²)/2. When tumors reached 200-300 mm³ mice were randomly separated into four groups and either left untreated (Control) or treated with doxorubicin (Doxo, 4 mg/kg/week administered intraperitoneally once weekly), with T12 (20 mg/kg/day diluted in drinking water daily), or with both (T12+Doxo).

For other assays, 10⁴ 4 T1 cells were suspended in 10 μl PBS and subcutaneously injected into the 4^{th} mammary fat pad of 4-week-old Balb/c female mice, and either left untreated or treated since the inoculation day with T12 (12 mg/kg/day in drinking water). Primary tumors formed and dimensions were periodically measured with a caliper. Metastases in lungs and spinal cord appeared several days after inoculation and could be monitored by PET with ¹⁸F-Fludeoxyglucose (radioactive glucose) using a Micro PET/CT (ALBIRA ARS). Mice were sacrificed (day 25^{th}), lungs were dissected and stained with Bouin's solution and metastases were counted. Where indicated, blood was collected and seeded in presence of 6-thioguanine and 4T1 cells selected and further cultured.

For still other assays, 10⁴ LLC cells suspended in PBS were injected subcutaneously into the right rear flank of 8-weeks GPBP-1^{-/-} (B6.129S(C)-Col4a3bp^{tm1.1Jsau}/Cnbc) or wild type C57BL/6 mice. The presence of tumors was detected by palpation of the skin. At day 28^{th} mice were sacrificed and the tumor removed. Lungs were excised, stained with Bouin's solution and analyzed to determine the presence of metastases. In each group, mice were classified as “with tumor” or “without tumor” or “with metastasis” or “without metastasis”. The statistical signification of the differences observed among groups was assessed by Fisher's exact test.

For some purposes, T12 was substituted by anti-GPBP N26 antibodies (1 mg/kg/week, intraperitoneal injection, once weekly).

### RNA Extraction and Gene Expression Analysis

Gene expression analyses were performed with human lung cancer A427 and A549 cells, circulating 4T1 mouse breast cancer cells isolated from blood of mice bearing 4T1 xenograft tumors, and 4T1 cells. Total RNA was extracted with Illustra RNASPIN® Mini (GE Healthcare) following manufacturer's instructions. Reverse transcription of 2-μg RNA samples was performed with High Capacity cDNA Reverse Transcription Kit (Applied Biosystems), and coupled qPCR analyses were performed with TAQMAN® Gene Expression Master Mix (Applied Biosystems) and specific TAQMAN® primers (Applied Biosystems) for human collagen IV genes (COL4A1, COL4A2, COL4A3, COL4A4, COL4A5 and COL4A6) and hypoxanthine-guanine phosphoribosyltransferase 1 (HPRT1) or mouse pbp (Col4a3bp), vimentin (Vim), E-cadherin (Cdh1) or hypoxanthine-guanine phosphoribosyltransferase 1 (Hprt1) genes, using a STEPONEPLUS® Real-Time PCR system (Applied Biosystems). HPRT1 and Hprt1 expression was used for reference purposes, and relative expressions were calculated with the ΔΔCt method. Duplicated runs were performed and the average ΔΔCt was used for calculations.

### Confocal Microscopy Studies

A549 spheroids were fixed with 4% formaldehyde in PBS (30 min, room temperature), rinsed twice with PBS, permeabilized with 0.2% Triton X-100 in PBS (5 min, room temperature) and blocked with 3% BSA in PBS (30 min, room temperature). Then spheroids were overnight incubated with suitable fluorophore-labeled primary antibodies diluted in blocking solution at 4° C., in a test tube with gentle rocking using a rotator. Spheroids were recovered by centrifugation (100×g, 5 min), washed with PBS and mounted for observation. For some purposes, spheroids were cultured in presence of 1 μM doxorubicin 3 hours before fixation.

Mouse tissue samples were frozen embedded in OCT (Sakura) and 5-μm-wide cryosections were prepared with a cryostat (Microm) and placed on crystal slides. Then samples were fixed with ice-cold 100% acetone for 10 min, washed with PBS and blocked first with 2.5% horse serum (Vector Laboratories) in PBS and then with avidin/biotin blocking kit (Vector Laboratories). For staining with biotinylated T12 (bioT12), ALEXA® Fluor 488-conjugated streptavidin (Invitrogen) was 1/500-diluted in a 130-μM bioT12 solution prepared in ENVISION® Flex Antibody Diluent (Dako) and incubated during 2 h. For additional staining fluorophore-labeled specific antibodies were added to staining mixtures. For confirmation of the specificity of bioT12 binding, 2.5 mM T12 was added as competing agent to the staining mixture (not shown). Cryosections were overnight incubated with staining mixtures in humid chamber at 4° C., washed with PBS and mounted for observation

For detection of collagen IV, anti-α1α2(IV) polyclonal antibodies (Merck Millipore) were labeled with ALEXA® Fluor® 647 Antibody Labeling Kit (Thermo Fisher Scientific). For detection of GPBP, anti-GPBP mAb el1.2 (Fibrostatin, SL) monoclonal antibodies were labeled with Pierce FITC Antibody Labeling Kit (Thermo Fisher Scientific), and N27 monoclonal antibodies with ALEXA® Fluor 546 Antibody Labeling Kit (Thermo Fisher Scientific).

For nuclei staining of spheroids or tissue sections, DAPI was added to antibody solutions. Observation of stained samples was performed with an Olympus FV1000 confocal microscope (Olympus) assembled on a motorized inverted IX8 microscope or a TCS-SP2 laser-scanning confocal spectral microscope (Leica) assembled to a Leica DM1RB inverted microscope.

Where indicated the Mean of fluorescence (±SEM) and the amount of pixels of images was measured using ADOBE PHOTOSHOP CS®. Co-localization studies were performed using WCIF ImageJ software. Output images only show co-localization points where the intensity of both fluorescent signals is above a pre-selected threshold.

### RNA Interference

SILENCER® Select siRNAs for COL4A3BP and COL4A1 and Silencer® Select Negative Control siRNA No. 1 (Thermo Fisher Scientific) were used to transfect A549 cells using LIPOFECTAMINE® 2000 reagent (Thermo Fisher Scientific) following manufacturer's indications.

### Western Blot Studies

For Western blot analysis, cells were lysed in TBS supplemented with 1% Triton X-100, 0.1% SDS, 1 mM PMSF, 10 μg/mL leupeptine and 20 mM NaF during 30 min at 4° C. Lysates were centrifuged (16,000×g, 5 min, 4° C.) and supernatants were collected and protein concentration determined (Bio-Rad Protein Assay). Samples were subjected to reducing SDS-PAGE and electro-transference onto PVDF membrane. Membranes were blocked in 5% skim milk in TBST and incubated with suitable primary antibodies and HRP-labeled secondary antibodies. Development was performed by chemiluminescence (ECL Prime, GE Healthcare) using an ImageQuant LAS 4000 Mini system (GE Healthcare). Mouse monoclonal N26, N27 and mAb e11-2 antibodies against GPBP were developed by Fibrostatin, S. L. Mouse monoclonal antibody against E-cadherin and rabbit monoclonal antibody against vimentin were purchased from Abcam. Mouse monoclonal antibody against GAPDH was gifted by Erwin Knecht. Rabbit polyclonal antibodies against phospho Thr 389 p70 S6 kinase and against active caspase 3 were from Cell Signaling Technology and Abcam, respectively.

Synthesis of bioT12

We developed a strategy to label T12 with essential vitamin (D-biotin). The Scheme 1 shows the synthesis of an adduct between T12 and D-biotin with the use of commercial 4-(Boc-amino)butyl bromide as a linker. The position chosen to anchor the linker to T12 was the methyl ether of compound 1 (described at U.S. Pat. No. 8,586,776B2) and the new conjugate obtained (5) was shown to inhibit GPBP kinase activity (data not shown) and used for tissue staining.

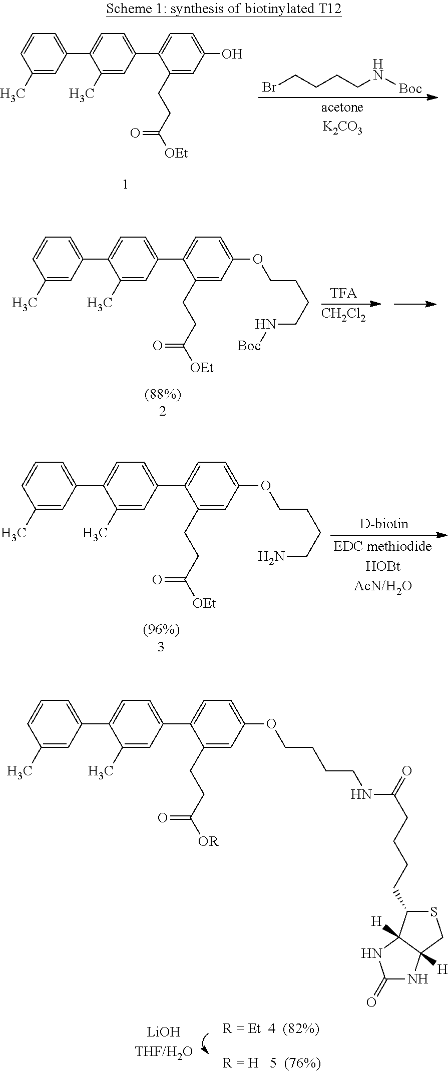

1. A method for inhibiting mesenchymal phenotype after epithelial-to-mesenchymal transition (EMT), or for treating an invasive tumor, comprising administering to a subject in need thereof an amount effective to inhibit mesenchymal phenotype after EMT, or to treat an invasive tumor, of an antibody selective for Goodpasture Antigen Binding Protein (GPBP), or a compound of formula: 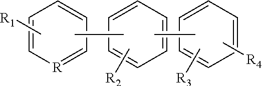
or a pharmaceutically acceptable salt thereof, wherein:
R is selected from N and CR₅;
R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, or (heteroaryl)C₁-C₆ alkyl;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, —O(CH₂)₁₋₅—C(O)(C₁-C₆ alkoxy), (aryl)C₁-C₆ alkyl, or (heteroaryl)C₁-C₆ alkyl.

2. The method of claim 1 wherein the compound is selected from the group consisting of:
ethyl (E)-3-[4″-(benzyloxy)-2′-formyl-3-methyl-(1,1′;4′,1″)terphenyl-2″-yl]acrylate;
ethyl 3-[4″-hydroxy-2′-(hydroxymethyl)-3-methyl-(1,1′;4′,1″)terphenyl-2″-yl]propionate;
3-[4″-hydroxy-2′-(hydroxymethyl)-3-methyl-(1,1′;4′,1″)terphenyl-2″-yl]propionic acid;
ethyl 3-[2′-(fluoromethyl)-4″-hydroxy-3-methyl-(1,1′;4′,1″)terphenyl-2″-yl]propionate;
ethyl 3-[2′-(hydroxymethyl)-4″-metoxy-3-methyl-(1,1′;4′,1″)terphenyl-2″-yl]propionate;
3-[4″-hydroxy-2′-(hydroxymethyl)-3-(trifluoromethyl)-(1,1′;4′,1″)terphenyl-2″-yl]propionic acid;
3-[4-hydroxy-3′-(hydroxymethyl)-4′-(pyridin-3-yl)biphenyl-2-yl]propionic acid;
3-[4″-hydroxy-2″-isopropyl-3-methyl-(1,1′;4′,1″)terphenyl-2′-yl]propionic acid;
(E)-ethyl 3-[4″-(benzyloxy)-2′-formyl-3-(trifluoromethyl)-(1,1′;4′,1″)terphenyl-2″-yl]acrylate;
(E)-ethyl 3-[4-(benzyloxy)-3′-formyl-4′-(pyridin-3-yl)biphenyl-2-yl]acrylate;
ethyl 3-[4″-hydroxy-2′,3-dimethyl-(1,1′;4′,1″)terphenyl-2″-yl]propionate;
ethyl 3-[4″-hydroxy-2′-methyl-3-(trifluoromethyl)-(1,1′;4′,1″)terphenyl-2″-yl]propionate;
3-[4″-hydroxy-2′,3-dimethyl-(1,1′;4′,1″)terphenyl-2″-yl]propionic acid;
3-[4″-hydroxy-2′-methyl-3-(trifluoromethyl)-(1,1′;4′,1″)terphenyl-2″-yl]propionic acid;
ethyl 3-[4″-hydroxy-2′-(hydroxymethyl)-3-(trifluoromethyl)-(1,1′;4′,1″)terphenyl-2″-yl]propionate;
ethyl 3-[4-hydroxy-3′-(hydroxymethyl)-4′-(pyridin-3-yl)biphenyl-2-yl]propionate;
ethyl 3-[2′-(fluoromethyl)-4″-hydroxy-3-(trifluoromethyl)-(1,1′;4′,1″)terphenyl-2″-yl]propionate;
ethyl 3-[3′-(fluoromethyl)-4-hydroxy-4′-(pyridin-3-yl)biphenyl-2-yl]propionate;
3-[2′-(fluoromethyl)-4″-hydroxy-3-methyl-(1,1′;4′,1″)terphenyl-2″-yl]propionic acid;
3-[2′-(fluoromethyl)-4″-hydroxy-3-(trifluoromethyl)-(1,1′;4′,1″)terphenyl-2″-yl]propionic acid;
3-[3′-(fluoromethyl)-4-hydroxy-4′-(pyridin-3-yl)biphenyl-2-yl]propionic acid;
ethyl (E)-3-[4″-(benzyloxy)-2′-(difluoromethyl)-3-methyl-(1,1′;4′,1″)terphenyl-2″-yl]acrylate;
ethyl (E)-3-[4″-(benzyloxy)-2′-(difluoromethyl)-3-(trifluoromethyl)-(1,1′;4′,1″)terphenyl-2″-yl]acrylate;
(E)-ethyl 3-[4-(benzyloxy)-3′-(difluoromethyl)-4′-(pyridin-3-yl)biphenyl-2-yl]acrylate;
ethyl 3-[2′-(difluoromethyl)-4″-hydroxy-3-methyl-(1,1′;4′,1″)terphenyl-2″-yl]propionate;
ethyl 3-[2′-(difluoromethyl)-4″-hydroxy-3-(trifluoromethyl)-(1,1′;4′,1″)terphenyl-2″-yl]propionate;
ethyl 3-[3′-(difluoromethyl)-4-hydroxy-4′-(pyridin-3-yl)biphenyl-2-yl]propionate;
3-[2′-(difluoromethyl)-4″-hydroxy-3-methyl-(1,1′;4′,1″)terphenyl-2″-yl]propionic acid;
3-[2′-(difluoromethyl)-4″-hydroxy-3-(trifluoromethyl)-(1,1′;4′,1″)terphenyl-2″-yl]propionic acid;
3-[3′-(difluoromethyl)-4-hydroxy-4′-(pyridin-3-yl)biphenyl-2-yl]propionic acid;
ethyl 3-[4″-methoxy-3,2′-dimethyl-(1,1′;4′,1″)terphenyl-2″-yl]propionate;
ethyl 3-[2′-(fluoromethyl)-4″-metoxy-3-methyl-(1,1′;4′,1″)terphenyl-2″-yl]propionate;
ethyl 3-[2′-(difluoromethyl)-4″-metoxy-3-methyl-(1,1′;4′,1″)terphenyl-2″-yl]propionate;
ethyl 3-[2′-(hydroxymethyl)-4″-metoxy-3-(trifluoromethyl)-(1,1′;4′,1″)terphenyl-2″-yl]propionate;
ethyl 3-[2′-methyl-4″-metoxy-3-(trifluoromethyl)-(1,1′;4′,1″)terphenyl-2″-yl]propionate;
ethyl 3-[2′-(fluoromethyl)-4″-metoxy-3-(trifluoromethyl)-(1,1′;4′,1″)terphenyl-2″-yl]propionate;
ethyl 3-[2′-(difluoromethyl)-4″-metoxy-3-(trifluoromethyl)-(1,1′;4′,1″)terphenyl-2″-yl]propionate;
ethyl 3-[3′-(hydroxymethyl)-4-metoxy-4′-(pyridin-3-yl)biphenyl-2-yl]propionate;
ethyl 3-[4-methoxy-3′-methyl-4′-(pyridin-3-yl)biphenyl-2-yl]propionate;
ethyl 3-[3′-(fluoromethyl)-4-metoxy-4′-(pyridin-3-yl)biphenyl-2-yl]propionate;
ethyl 3-[3′-(difluoromethyl)-4-metoxy-4′-(pyridin-3-yl)biphenyl-2-yl]propionate;
3-[2′-(hydroxymethyl)-4″-methoxy-3-methyl-(1,1′;4′,1″)terphenyl-2″-yl]propionic acid;
3-[4″-methoxy-3,2′-dimethyl-(1,1′;4′,1″)terphenyl-2″-yl]propionic acid;
3-[2′-(fluoromethyl)-4″-methoxy-3-methyl-(1,1′;4′,1″)terphenyl-2″-yl]propionic acid;
3-[2′-(difluoromethyl)-4″-methoxy-3-methyl-(1,1′;4′,1″)terphenyl-2″-yl]propionic acid;
3-[2′-(hydroxymethyl)-4″-methoxy-3-(trifluoromethyl)-(1,1′;4′,1″)terphenyl-2″-yl]propionic acid;
3-[2′-methyl-4″-methoxy-3-(trifluoromethyl)-(1,1′;4′,1″)terphenyl-2″-yl]propionic acid;
3-[2′-(fluoromethyl)-4″-methoxy-3-(trifluoromethyl)-(1,1′;4′,1″)terphenyl-2″-yl]propionic acid;
3-[2′-(difluoromethyl)-4″-methoxy-3-(trifluoromethyl)-(1,1′;4′,1″)terphenyl-2″-yl]propionic acid;
3-[3′-(hydroxymethyl)-4-methoxy-4′-(pyridin-3-yl)-biphenyl-2-yl]propionic acid;
3-[4-methoxy-3′-methyl-4′-(pyridin-3-yl)biphenyl-2-yl]propionic acid;
3-[3′-(fluoromethyl)-4-methoxy-4′-(pyridin-3-yl)-biphenyl-2-yl]propionic acid;
3-[3′-(difluoromethyl)-4-methoxy-4′-(pyridin-3-yl)-biphenyl-2-yl]propionic acid;
ethyl 3-[3,2′-dimethyl-4″-propoxy-(1,1′;4′,1″)terphenyl-2″-yl]propionate;
ethyl 3-[4″-(ethoxycarbonylmethoxy)-3,2′-dimethyl-(1,1′;4′,1″)terphenyl-2″-yl]propionate;
ethyl 3-[2′-methyl-4″-propoxy-3-(trifluoromethyl)-(1,1′;4′,1″)terphenyl-2″-yl]propionate;
3-[3,2′-dimethyl-4″-propoxy-(1,1′;4′,1″)terphenyl-2″-yl]propionic acid;
3-[4″-(carboxymethoxy)-3,2′-dimethyl-(1,1′;4′,1″)terphenyl-2″-yl]propionic acid;
3-[2′-methyl-4″-propoxy-3-(trifluoromethyl)-(1,1′;4′,1″)terphenyl-2″-yl]propionic acid;
ethyl 3-[3′-formyl-4-metoxy-4′-(pyridin-3-yl)biphenyl-2-yl]propionate;
ethyl 3-[4,4″-dimethoxy-3,2′-dimethyl-(1,1′;4′,1″)terphenyl-2″-yl]propionate;
3-[4,4″-dimethoxy-3,2′-dimethyl-(1,1′;4′,1″)terphenyl-2″-yl]propionic acid;
ethyl (E)-3-[4″-(benzyloxy)-3-formyl-2″-isopropyl-(1,1′;4′,1″)terphenyl-2′-yl]acrylate;
ethyl 3-[4″-hydroxy-2″-isopropyl-3-methyl-(1,1′;4′,1″)terphenyl-2′-yl]propionate;
3-[3-chloro-2′-methyl-4,4″-dimethoxy-(1,1′;4′,1″)terphenyl-2″-yl]propionic acid;
or a pharmaceutically acceptable salt thereof.

3. The method of claim 1, wherein the compound is 3-[4″-methoxy-3,2′-dimethyl-(1,1′;4′,1″)terphenyl-2″-yl] propionic acid, or a pharmaceutically acceptable salt thereof.

4. The method of claim 1, wherein the method is for inhibiting mesenchymal phenotype after EMT, and wherein the subject has a disorder selected from the group consisting of chronic kidney disease immune complex mediated glomerulonephritis, organ fibrosis, pulmonary fibrosis, rheumatoid arthritis, and in invasive tumor.

5. The method of claim 1, wherein the subject has an altered expression of cell markers in a relevant tissue sample compared to a control tissue sample, wherein the altered expression is indicative of an epithelial-to-mesenchymal phenotype transition.

6. The method of claim 5, wherein the cell markers include one or more of vimentin, E-cadherin, collagens I and IV, MMP-9, CCL2/MCP-1, α5 (IV) chain, (α5 (IV))₃ protomer, and Goodpasture antigen binding protein (GPBP).

7. The method of claim 6, wherein the subject has an increase in vimentin expression and a decrease in E-cadherin expression in a relevant tissue sample compared to an epithelial cell control.

8. The method of claim 1, wherein the subject has an increased expression of α5(IV) chain, and/or (α5 (IV))₃ protomer in a relevant tissue sample compared to a control tissue sample, wherein the increase expression is indicative of an epithelial-to-mesenchymal phenotype transition and/or an invasive tumor phenotype.

9. The method of claim 8, wherein the subject also has an increased expression of (α1)₂α2 (IV) protomer and/or an increased expression α1,α2 (IV) chains in a relevant tissue sample compared to a control tissue sample, wherein the increase expression is indicative of an epithelial-to-mesenchymal phenotype transition and/or an invasive tumor phenotype

10. The method of claim 1, wherein the method is for treating an invasive tumor, and wherein the invasive tumor is an invasive carcinoma.

11. The method of claim 10, wherein the invasive carcinoma is selected from the group consisting of an invasive breast tumor and an invasive lung tumor.

12. The method of claim 1, wherein the method is for treating an invasive tumor, and wherein treating the invasive tumor reduces tumor metastases in the subject.

13. The method of claim 1, wherein the compound is the only therapeutic administered to the subject.

14. A method for detecting EMT in a tissue, comprising
(a) contacting a tissue in a subject with an amount effective to label the tissue of a detectably labeled compound of formula: 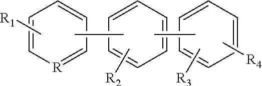
or a pharmaceutically acceptable salt thereof, wherein:
R is selected from N and CR₅;
R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, or (heteroaryl)C₁-C₆ alkyl;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, —O(CH₂)₁₋₅—C(O)(C₁-C₆ alkoxy), (aryl)C₁-C₆ alkyl, or (heteroaryl)C₁-C₆ alkyl;
for a time and under conditions suitable to promote binding of the detectably labeled compound to the tissue; and
(b) detecting the detectably labeled compound bound to the tissue, thereby detecting EMT in the tissue,

15. The method of claim 14 wherein the compound is selected from the group consisting of:
ethyl (E)-3-[4″-(benzyloxy)-2′-formyl-3-methyl-(1,1′;4′,1″)terphenyl-2″-yl]acrylate;
ethyl 3-[4″-hydroxy-2′-(hydroxymethyl)-3-methyl-(1,1′;4′,1″)terphenyl-2″-yl]propionate;
3-[4″-hydroxy-2′-(hydroxymethyl)-3-methyl-(1,1′;4′,1″)terphenyl-2″-yl]propionic acid;
ethyl 3-[2′-(fluoromethyl)-4″-hydroxy-3-methyl-(1,1′;4′,1″)terphenyl-2″-yl]propionate;
ethyl 3-[2′-(hydroxymethyl)-4″-metoxy-3-methyl-(1,1′;4′,1″)terphenyl-2″-yl]propionate;
3-[4″-hydroxy-2′-(hydroxymethyl)-3-(trifluoromethyl)-(1,1′;4′,1″)terphenyl-2″-yl]propionic acid;
3-[4-hydroxy-3′-(hydroxymethyl)-4′-(pyridin-3-yl)biphenyl-2-yl]propionic acid;
3-[4″-hydroxy-2″-isopropyl-3-methyl-(1,1′;4′,1″)terphenyl-2′-yl]propionic acid;
(E)-ethyl 3-[4″-(benzyloxy)-2′-formyl-3-(trifluoromethyl)-(1,1′;4′,1″)terphenyl-2″-yl]acrylate;
(E)-ethyl 3-[4-(benzyloxy)-3′-formyl-4′-(pyridin-3-yl)biphenyl-2-yl]acrylate;
ethyl 3-[4″-hydroxy-2′,3-dimethyl-(1,1′;4′,1″)terphenyl-2″-yl]propionate;
ethyl 3-[4″-hydroxy-2′-methyl-3-(trifluoromethyl)-(1,1′;4′,1″)terphenyl-2″-yl]propionate;
3-[4″-hydroxy-2′,3-dimethyl-(1,1′;4′,1″)terphenyl-2″-yl]propionic acid;
3-[4″-hydroxy-2′-methyl-3-(trifluoromethyl)-(1,1′;4′,1″)terphenyl-2″-yl]propionic acid;
ethyl 3-[4″-hydroxy-2′-(hydroxymethyl)-3-(trifluoromethyl)-(1,1′;4′,1″)terphenyl-2″-yl]propionate;
ethyl 3-[4-hydroxy-3′-(hydroxymethyl)-4′-(pyridin-3-yl)biphenyl-2-yl]propionate;
ethyl 3-[2′-(fluoromethyl)-4″-hydroxy-3-(trifluoromethyl)-(1,1′;4′,1″)terphenyl-2″-yl]propionate;
ethyl 3-[3′-(fluoromethyl)-4-hydroxy-4′-(pyridin-3-yl)biphenyl-2-yl]propionate;
3-[2′-(fluoromethyl)-4″-hydroxy-3-methyl-(1,1′;4′,1″)terphenyl-2″-yl]propionic acid;
3-[2′-(fluoromethyl)-4″-hydroxy-3-(trifluoromethyl)-(1,1′;4′,1″)terphenyl-2″-yl]propionic acid;
3-[3′-(fluoromethyl)-4-hydroxy-4′-(pyridin-3-yl)biphenyl-2-yl]propionic acid;
ethyl (E)-3-[4″-(benzyloxy)-2′-(difluoromethyl)-3-methyl-(1,1′;4′,1″)terphenyl-2″-yl]acrylate;
ethyl (E)-3-[4″-(benzyloxy)-2′-(difluoromethyl)-3-(trifluoromethyl)-(1,1′;4′,1″)terphenyl-2″-yl]acrylate;
(E)-ethyl 3-[4-(benzyloxy)-3′-(difluoromethyl)-4′-(pyridin-3-yl)biphenyl-2-yl]acrylate;
ethyl 3-[2′-(difluoromethyl)-4″-hydroxy-3-methyl-(1,1′;4′,1″)terphenyl-2″-yl]propionate;
ethyl 3-[2′-(difluoromethyl)-4″-hydroxy-3-(trifluoromethyl)-(1,1′;4′,1″)terphenyl-2″-yl]propionate;
ethyl 3-[3′-(difluoromethyl)-4-hydroxy-4′-(pyridin-3-yl)biphenyl-2-yl]propionate;
3-[2′-(difluoromethyl)-4″-hydroxy-3-methyl-(1,1′;4′,1″)terphenyl-2″-yl]propionic acid;
3-[2′-(difluoromethyl)-4″-hydroxy-3-(trifluoromethyl)-(1,1′;4′,1″)terphenyl-2″-yl]propionic acid;
3-[3′-(difluoromethyl)-4-hydroxy-4′-(pyridin-3-yl)biphenyl-2-yl]propionic acid;
ethyl 3-[4″-methoxy-3,2′-dimethyl-(1,1′;4′,1″)terphenyl-2″-yl]propionate;
ethyl 3-[2′-(fluoromethyl)-4″-metoxy-3-methyl-(1,1′;4′,1″)terphenyl-2″-yl]propionate;
ethyl 3-[2′-(difluoromethyl)-4″-metoxy-3-methyl-(1,1′;4′,1″)terphenyl-2″-yl]propionate;
ethyl 3-[2′-(hydroxymethyl)-4″-metoxy-3-(trifluoromethyl)-(1,1′;4′,1″)terphenyl-2″-yl]propionate;
ethyl 3-[2′-methyl-4″-metoxy-3-(trifluoromethyl)-(1,1′;4′,1″)terphenyl-2″-yl]propionate;
ethyl 3-[2′-(fluoromethyl)-4″-metoxy-3-(trifluoromethyl)-(1,1′;4′,1″)terphenyl-2″-yl]propionate;
ethyl 3-[2′-(difluoromethyl)-4″-metoxy-3-(trifluoromethyl)-(1,1′;4′,1″)terphenyl-2″-yl]propionate;
ethyl 3-[3′-(hydroxymethyl)-4-metoxy-4′-(pyridin-3-yl)biphenyl-2-yl]propionate;
ethyl 3-[4-methoxy-3′-methyl-4′-(pyridin-3-yl)biphenyl-2-yl]propionate;
ethyl 3-[3′-(fluoromethyl)-4-metoxy-4′-(pyridin-3-yl)biphenyl-2-yl]propionate;
ethyl 3-[3′-(difluoromethyl)-4-metoxy-4′-(pyridin-3-yl)biphenyl-2-yl]propionate;
3-[2′-(hydroxymethyl)-4″-methoxy-3-methyl-(1,1′;4′,1″)terphenyl-2″-yl]propionic acid;
3-[4″-methoxy-3,2′-dimethyl-(1,1′;4′,1″)terphenyl-2″-yl]propionic acid;
3-[2′-(fluoromethyl)-4″-methoxy-3-methyl-(1,1′;4′,1″)terphenyl-2″-yl]propionic acid;
3-[2′-(difluoromethyl)-4″-methoxy-3-methyl-(1,1′;4′,1″)terphenyl-2″-yl]propionic acid;
3-[2′-(hydroxymethyl)-4″-methoxy-3-(trifluoromethyl)-(1,1′;4′,1″)terphenyl-2″-yl]propionic acid;
3-[2′-methyl-4″-methoxy-3-(trifluoromethyl)-(1,1′;4′,1″)terphenyl-2″-yl]propionic acid;
3-[2′-(fluoromethyl)-4″-methoxy-3-(trifluoromethyl)-(1,1′;4′,1″)terphenyl-2″-yl]propionic acid;
3-[2′-(difluoromethyl)-4″-methoxy-3-(trifluoromethyl)-(1,1′;4′,1″)terphenyl-2″-yl]propionic acid;
3-[3′-(hydroxymethyl)-4-methoxy-4′-(pyridin-3-yl)-biphenyl-2-yl]propionic acid;
3-[4-methoxy-3′-methyl-4′-(pyridin-3-yl)biphenyl-2-yl]propionic acid;
3-[3′-(fluoromethyl)-4-methoxy-4′-(pyridin-3-yl)-biphenyl-2-yl]propionic acid;
3-[3′-(difluoromethyl)-4-methoxy-4′-(pyridin-3-yl)-biphenyl-2-yl]propionic acid;
ethyl 3-[3,2′-dimethyl-4″-propoxy-(1,1′;4′,1″)terphenyl-2″-yl]propionate;
ethyl 3-[4″-(ethoxycarbonylmethoxy)-3,2′-dimethyl-(1,1′;4′,1″)terphenyl-2″-yl]propionate;
ethyl 3-[2′-methyl-4″-propoxy-3-(trifluoromethyl)-(1,1′;4′,1″)terphenyl-2″-yl]propionate;
3-[3,2′-dimethyl-4″-propoxy-(1,1′;4′,1″)terphenyl-2″-yl]propionic acid;
3-[4″-(carboxymethoxy)-3,2′-dimethyl-(1,1′;4′,1″)terphenyl-2″-yl]propionic acid;
3-[2′-methyl-4″-propoxy-3-(trifluoromethyl)-(1,1′;4′,1″)terphenyl-2″-yl]propionic acid;
ethyl 3-[3′-formyl-4-metoxy-4′-(pyridin-3-yl)biphenyl-2-yl]propionate;
ethyl 3-[4,4″-dimethoxy-3,2′-dimethyl-(1,1′;4′,1″)terphenyl-2″-yl]propionate;
3-[4,4″-dimethoxy-3,2′-dimethyl-(1,1′;4′,1″)terphenyl-2″-yl]propionic acid;
ethyl (E)-3-[4″-(benzyloxy)-3-formyl-2″-isopropyl-(1,1′;4′,1″)terphenyl-2′-yl]acrylate;
ethyl 3-[4″-hydroxy-2″-isopropyl-3-methyl-(1,1′;4′,1″)terphenyl-2′-yl]propionate;
3-[3-chloro-2′-methyl-4,4″-dimethoxy-(1,1′;4′,1″)terphenyl-2″-yl]propionic acid;
or a pharmaceutically acceptable salt thereof.

16. The method of claim 14, wherein the compound is 3-[4″-methoxy-3,2′-dimethyl-(1,1′;4′,1″)terphenyl-2″-yl] propionic acid, or a pharmaceutically acceptable salt thereof.

17. The method of claim 14, wherein the tissue is selected from the group consisting of a tumor, a joint, and tissue from any organ.

18. The method of claim 17, wherein one of the following is true:
(a) the tissue is a kidney, and detecting EMT in the kidney indicates that the subject has chronic kidney disease or immune-complex mediated glomerulonephritis.
(b) the tissue is tissue from any organ, and wherein detecting EMT indicates that the subject has organ fibrosis.
(c) the tissue is a lung, and wherein detecting EMT in the lung indicates that the subject has pulmonary fibrosis; or.
(d) the tissue is a joint, and wherein detecting EMT indicates that the subject has rheumatoid arthritis.

19. The method of claim 17, wherein the tissue is a tumor, and wherein detecting EMT indicates that the subject has an invasive tumor.

20. The method of claim 19 wherein the tumor is an invasive carcinoma.

21. The method of claim 20, wherein the invasive carcinoma is selected from the group consisting of an invasive breast tumors and an invasive lung tumor.
